(19) United States
(12) Patent Application Publication
(10) Pub. No.: US 2024/0301218 A1
(43) Pub. Date: Sep. 12, 2024

(54) Polymeric Antifouling Coating with Antimicrobial Peptides

(71) Applicant: THE UNIVERSITY OF BRITISH COLUMBIA, Vancouver, BC (CA)

(72) Inventors: Jayachandran N. Kizhakkedathu, Vancouver, BC (CA); Dirk Lange, Vancouver, BC (CA); Kai Yu, Vancouver, BC (CA); Robert E.W. Hancock, Vancouver, BC (CA)

(21) Appl. No.: 18/565,049

(22) PCT Filed: Jun. 2, 2022
(86) PCT No.: PCT/CA2022/050883

§ 371 Date: Nov. 28, 2023

Related U.S. Application Data
(60) Provisional application No. 63/195,836, filed on Jun. 2, 2021.

(51) Int. Cl. C09D 5/14; A01N 37/46; A01P 1/00; A61L 29/08; A61L 29/16; C09D 5/16; C09D 133/26; C09D 165/00

(52) U.S. Cl. CPC C09D 5/14; A01N 37/46; A01P 1/00; A61L 29/085; A61L 29/16; C09D 5/16; C09D 133/26; C09D 165/00; A61L 2300/404; A61L 2300/424

(57) ABSTRACT

Provided herein are compositions including polymeric binder, for example polydopamine (PDA); a poly(N,N-dimethylacrylamide) (PDMA) polymer or a PDMA co-N-(3-Aminopropyl) Methacrylamide (APMA) polymer, and an antimicrobial peptide (AMP), methods for using the compositions to coat a substrate, and methods for making the compositions. Alternatively, the composition may include a polymeric binder or a salt thereof, high molecular weight polymer and a pharmaceutically active agent. In particular, the substrate may form part of an apparatus on which it would be beneficial to limit biofouling and/or protein binding.

### CROSS REFERENCE TO RELATED APPLICATIONS

This application claims the benefit of U.S. Provisional Patent Application Ser. No. 63/195,836 filed on 2 Jun. 2021 entitled “HIGH EFFICIENCY ANTIFOULING COATING WITH ANTIMICROBIAL PEPTIDES”.

### TECHNICAL FIELD

This invention relates to substrate independent coatings utilizing self-assembly of dopamine or other polymeric binders; ultra-high or high molecular weight hydrophilic polymers; and conjugated antimicrobial peptides (AMPs); and methods for making such coatings. In particular, the invention relates to coatings that may be applied onto substrates, such as medical devices and implants.

### BACKGROUND

The onset of infections caused by opportunistic biofilm-forming pathogens can lead to severe infections and, in some cases, cause death, particularly in immunocompromised patients. Various approaches are currently being investigated to prevent implant/device associated infections, and the most promising approaches include both the prevention of bacterial adhesion and biofilm formation. Anti-adhesive and antimicrobial coatings have shown great promise to combat device/implant infections. These dual functional antibacterial surfaces are prepared by incorporating antimicrobial agents into non-fouling materials through covalent bonding^{[1-2]} or layer-by-layer (LBL) deposition^{[3-4]}. There are several configurations or structures that have been adopted in the literature, including combining biocidal sub-layers with antifouling upper layers^{[5-6]}, contact-active antimicrobial upper-layers and antifouling sub-layers^{[7-8]} and, layering with evenly mixed antimicrobial and non-fouling components^{[9-14]}. In principle, such surfaces should have both passive and active functions simultaneously, to improve the overall antibacterial efficacy.

With regards to antimicrobial agents, coatings containing covalently attached antimicrobial peptides (AMPs) showed excellent activity in preventing infections associated with implants in mouse models^{[14-16]}. AMPs have broad spectrum anti-biofilm activity and minimal chance of developing resistance due to the multiple modes of action of the AMPs. However, tethered AMPs often showed decreased activity when compared to their soluble forms^{[17]}, which limits the efficient biofilm prevention by most peptides. The conjugation methods also influence the activity of tethered peptides since they can interfere with the mobility and flexibility of tethered AMPs^{[17-23]}. Most often, the conjugation is dependent on the substrate (different biomedical plastics, metals, ceramics, hydrogels etc.), and multiple modification steps (and chemistry) are needed to achieve a stable AMP-based coating on the surface^{[17-23]}.

Mussel-inspired polydopamine-based coating provide a versatile platform to construct an antimicrobial coating on a variety of substrates. Antimicrobial peptides, including nisin^{[24]}, magainin II^{[25-26]}, synthetic antimicrobial peptide CWR11^{[27]}, cecropin B^{[28]}, SESB2V^{[29]}, and antimicrobial lipopeptide^{[30]} have been grafted to surfaces utilizing dopamine coating. Although such AMP conjugated coatings showed antibacterial efficiency, such coatings showed poor resistance to protein fouling, adhesion of dead/live bacteria, which results in diminished anti-biofilm activity in long-term and activity in relevant animal models. Inactivation of such technologies is driven by the accumulation of proteins and dead bacteria killed by contact killing on the surface providing a pedestal for subsequent biofilm formation by live bacteria. Adhered dead bacteria and proteins that collect on a surface provide good support for eventual biofilm formation leading to infection. Therefore, coatings with dual functionality, antifouling and antimicrobial, are more desirable to combat implant/device-related infections. Reches et al. designed an AMP with three moieties (adhesive dopamine moiety, antimicrobial and antifouling motifs) and achieved dual functionality that resists biofouling^{[31]}. Yang et al. achieved this functionality by conjugating both polylysine and copolymer with zwitterionic segments^{[32]}. However, it is challenging to apply such coatings onto a medical device or on an implant surface due to complicated synthesis and the need for multiple chemical modifications. Accordingly, it is not an ideal platform to optimize antimicrobial and antifouling properties in a coating.

Another consideration is that previous AMP screening studies for the identification of surface-active AMPs have been built on different solid supports (for example, resin beads, pins, glass chips, tea bags, and cellulose membranes) that did not take account of non-specific adhesion of bacteria which can foul the support surface. In these methods, the AMPs were tethered so that peptide density was maximized. However, in these studies the tethered peptide easily became overwhelmed by the planktonic bacteria, which resulted in a surface fouled by the dead and/or live bacteria at the surface^{[16-17, 33-35]}.

Furthermore, these surfaces tend to get easily fouled by proteins and cells present in complex biological environment. The short-term activity readouts from different assays are thus not a true measure of their long-term activity.

### SUMMARY

The present invention is based, in part, on the surprising discovery that some polymers or polymer combinations are more useful than others in coating substrates to both prevent fouling and have antibacterial activity. In particular, coating compositions are provided, wherein the coating composition includes a polydopamine (PDA); a poly(N,N-dimethylacrylamide) (PDMA) polymer or a PDMA co-N-(3-Aminopropyl) Methacrylamide (APMA) polymer; and an antimicrobial peptide (AMP). Alternatively, there are provided coating compositions where the PDMA-co-APMA is substituted with a hydrophilic polymer co-APMA polymer as described herein. Alternatively PDA may be substituted with another polymeric binder.

Having antimicrobial peptides (AMPs) attached in a non-fouling background has the potential to manifest peptide activity in an uncompromised manner with a greater possibility for success when choosing an implant or a medical device coating. Also, a coating method that is simple, substrate-independent, and capable of generating a non-fouling background could potentially be used as a method for screening AMPs for anti-biofilm activity in an environment that more closely approximates the environment that an implant or a medical device is likely to be used, and is thus, is more likely to produce successful candidates.

The present invention is based in part on the surprising discovery that specific conjugation methods to produce antifouling coatings are able to retain the activity of AMPs. Two methods of conjugation are described herein.

In a first embodiment there is provided a coating composition, the coating composition including: (a) a polydopamine (PDA); (b) a poly(N,N-dimethylacrylamide) (PDMA) polymer or a PDMA co-N-(3-Aminopropyl) Methacrylamide (APMA) polymer; and (c) an antimicrobial peptide (AMP).

In a further embodiment there is provided a coating composition, the coating composition including: (a) a polymeric binder; (b) a poly(N,N-dimethylacrylamide) (PDMA) polymer or a PDMA co-N-(3-Aminopropyl) Methacrylamide (APMA) polymer; and (c) an antimicrobial peptide (AMP).

In a further embodiment there is provided a coating composition, the coating composition including: (a) a polymeric binder; (b) a hydrophilic polymer, and (c) an antimicrobial peptide (AMP), wherein the hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

In a further embodiment there is provided a coating composition, the coating composition including: (a) a polymeric binder; (b) a hydrophilic polymer co-APMA polymer; and (c) an antimicrobial peptide (AMP), wherein the hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

In a further embodiment there is provided a coated substrate, the coated substrate including: (a) a substrate; (b) a polydopamine; (c) a PDMA polymer or a PDMA co-polymer; and (d) an AMP.

In a further embodiment there is provided a coated substrate, the coated substrate including: (a) a substrate; (b) a polymeric binder, (c) a PDMA polymer or a PDMA co-polymer; and (d) an AMP.

In a further embodiment there is provided a coated substrate, the coated substrate including: (a) a substrate; (b) a polymeric binder; (c) a hydrophilic polymer; and (d) an AMP, wherein the hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

In a further embodiment there is provided a coated substrate, the coated substrate including: (a) a substrate; (b) a polymeric binder; (c) a hydrophilic polymer co-APMA polymer; and (d) an AMP, wherein the hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

In a further embodiment there is provided a medical device including: a structure for implantation or disposition inside a subject, the structure including at least one surface for coating; wherein the at least one surface has a coating disposed directly on the at least one surface of the medical device, the coating comprising: (a) a polydopamine; (b) a PDMA polymer or a PDMA co-polymer, and (c) an antimicrobial peptide (AMP).

In a further embodiment there is provided a coating composition, the coating composition including: (a) a polydopamine (PDA); and (b) PDMA co-polymer. The coating composition may further include an antimicrobial peptide (AMP).

In a further embodiment there is provided a coating composition, the coating composition including: (a) a polydopamine (PDA); (b) a hydrophilic polymer co-APMA polymer; and (c) an antimicrobial peptide (AMP); wherein, the hydrophilic polymer co-polymer may have the structure of Formula I:

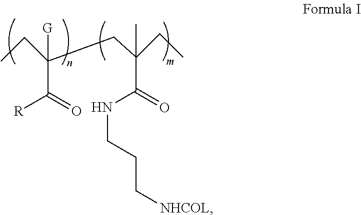

wherein R may be selected from

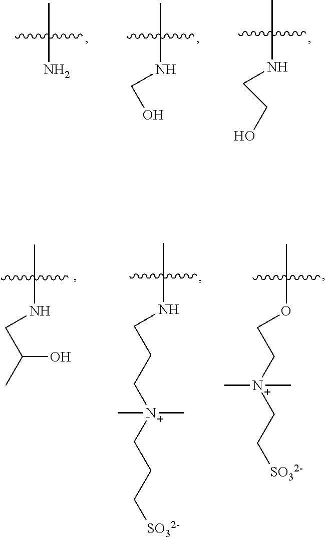

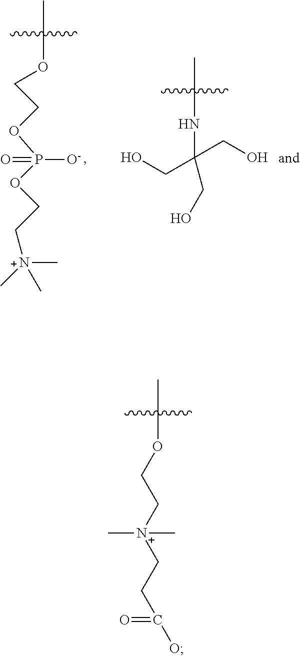

and G may be H or —CH₃; L may be a linking moiety; n may be an integer between woo and 20,000; and m may be an integer between 1 and 10,000. The linking moiety may be selected from: CH₂I, CH₂Br,

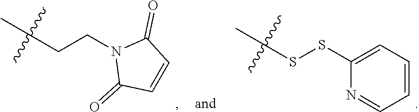 [SMILES: *CCN1C(=O)C=CC1=O ; *SSc1ccccn1]

The hydrophilic polymer may be copolymerized with: an Iodoacetyl Linker (co-APMA-I); a Bromoacetyl Linker (co-APMA-Br); a Maleimide Linker (co-APMA-M); or a Pyridyl disulfide Linker (co-APMA-Pd).

In a further embodiment there is provided a substrate coating method, the method including: (a) bringing the substrate into contact with a PDA and PDMA polymer or a PDMA co-APMA polymer solution; (b) rinsing and drying; (c) bringing the substrate into contact with an AMP solution; (d) adding of a thiol containing hydrophilic compound; and (e) rinsing and drying. The substrate coating method may further include a cleaning of the substrate prior to step (a).

In a further embodiment there is provided a method of coating a substrate, wherein the substrate is immersed in a solution including the coating composition as described herein.

In a further embodiment there is provided a method of coating a substrate, wherein the substrate is sprayed with a solution or solutions including the composition as described herein.

In a further embodiment there is provided a coating composition, the coating composition including: (a) a polymeric binder; (b) a hydrophilic polymer; and (c) an antimicrobial peptide (AMP); wherein hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

In a further embodiment there is provided a coating composition, the coating composition comprising: (a) a polymeric binder, (b) a hydrophilic polymer co-APMA polymer; and (c) an antimicrobial peptide (AMP); wherein, the hydrophilic polymer co-polymer has the structure of Formula I:

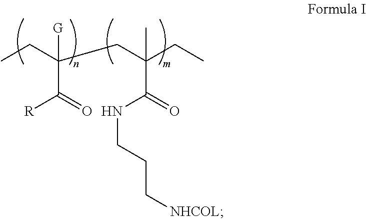

R may be selected from

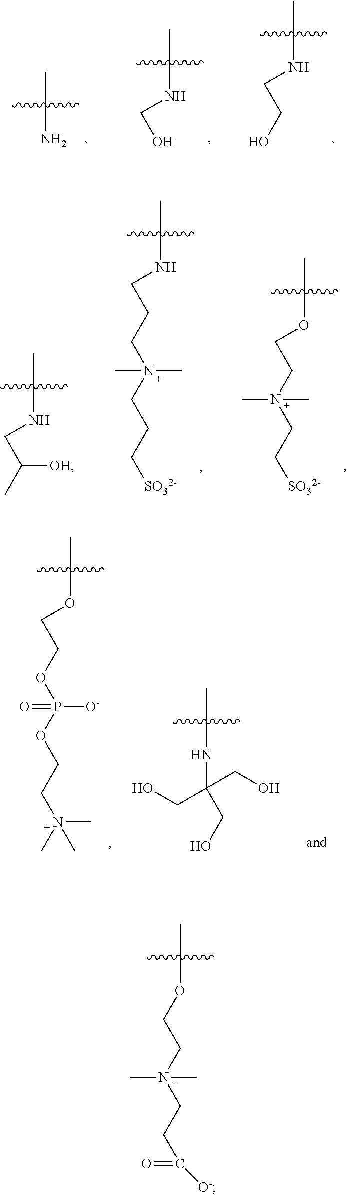

and G may be H or —CH₃; L may be a linking moiety; n may be an integer between 1000 and 20,000; and m is an integer between 1 and 10,000. The polymeric binder may be selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin. L may be CH₂I, CH₂Br,

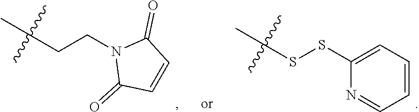 [SMILES: *CCN1C(=O)C=CC1=O ; *SSc1ccccn1]

The linking moiety may be CH₂I, CH₂Br,

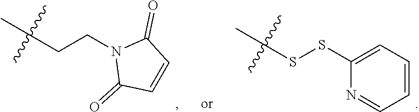 [SMILES: *CCN1C(=O)C=CC1=O ; *SSc1ccccn1]

n may be an integer between woo and 20,000. m may be an integer between 1 and 10,000. n may be an integer between 1,000 and 30,000. m may be an integer between 1 and 20,000. n may be an integer between 1,000 and 40,000. m may be an integer between 1 and 30,000. n may be an integer between 2,000 and 20,000. m may be an integer between 10 and 10,000. n may be an integer between 3,000 and 20,000. m may be an integer between 20 and 10,000. n may be an integer between 4,000 and 20,000. m may be an integer between 30 and 10,000. n may be an integer between 5,000 and 20,000. m may be an integer between 40 and 10,000. n may be an integer between 6,000 and 20,000. m may be an integer between 50 and 10,000. n may be an integer between 7,000 and 20,000. m may be an integer between 60 and 10,000. n may be an integer between 8,000 and 20,000. m may be an integer between 70 and 10,000. n may be an integer between 9,000 and 20,000. m may be an integer between 80 and 10,000. n may be an integer between 10,000 and 20,000. m may be an integer between 90 and 10,000. n may be an integer between 1,000 and 10,000. m may be an integer between 100 and 10,000. n may be an integer between 500 and 20,000. m may be an integer between 1,000 and 10,000. n may be an integer between 1 and 1000 and m may be an integer between 1 and 1000. n may be an integer between 1 and 900 and m may be an integer between 1 and 900. n may be an integer between 1 and 800 and m may be an integer between 1 and 800. n may be an integer between 1 and 700 and m may be an integer between 1 and 700. n may be an integer between 1 and 600 and m may be an integer between 1 and 600. n may be an integer between 1 and 500 and m may be an integer between 1 and 500. n may be an integer between 1 and 400 and m may be an integer between 1 and 400. n may be an integer between 1 and 300 and m may be an integer between 1 and 300. n may be an integer between 1 and 200 and m may be an integer between 1 and 200. n may be an integer between 1 and 100 and m may be an integer between 1 and 100.

In a further embodiment there is provided a substrate coating method, the method comprising: (a) bringing the substrate into contact with a hydrophilic polymer and a polymeric binder solution; (b) rinsing and drying; (c) bringing the substrate into contact with an AMP solution; (d) adding of a thiol containing hydrophilic compound; and (e) rinsing and drying. The polymeric binder may be selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin. The hydrophilic polymer may be selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate). The substrate coating method may further including a cleaning of the substrate prior to step (a).

The PDMA polymer may be either a high-molecular-weight (hPDMA) or an ultrahigh-molecular-weight (uhPDMA). The PDMA co-APMA may further include a linker. The linker may be selected from: an Iodoacetyl Linker (PDMA-co-APMA-I); a Bromoacetyl Linker (PDMA-co-APMA-Br); a Maleimide Linker (PDMA-co-APMA-M); and Pyridyl disulfide Linker (PDMA-co-APMA-Pd). The AMP may be selected from one or more of: E6; Tet20C; Tet20LC; DJK5C; DJK5; DJK6; RI-DJK5; IDR-1018; and 3002C. The AMP may be conjugated by an amine group or a thiol group to a quinone group on the PDA. The thiol groups on the AMP may also be conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer. The AMP may be E6. The AMP may be Tet20C. The AMP may be Tet20LC. The AMP may be DJK5C. The AMP may be DJK5. The AMP may be DJK6. The AMP may be RI-DJK5. The AMP may be IDR-1018. The AMP may be 3002C. The poly(N,N-dimethylacrylamide) (PDMA) polymer or the PDMA co-N-(3-Aminopropyl) Methacrylamide (APMA) polymer may be uhPDMA.

The coating composition may have anti-fouling activity and antimicrobial activity. The coating composition may have anti-adhesion activity. The coating composition may be for use in coating a medical device. The medical device may be for implantation in a subject.

The substrate may be selected from: a plastic; a metal; a ceramic; a carbon based material; a metal oxide; a hydrogels; a biological tissue; a wood; a cement; a rubber, a resin; and a composite. The substrate may be selected from: poly(propylene) (PP); poly(urethane) (PU); poly(ethylene) (PE); unplasticized polyvinyl chloride (uPVC); plasticized polyvinyl chloride (pPVC); poly(imide) (PI); ethylene vinyl acetate (EVA); poly(tetrafluoroethylene) (PTFE); polydimethylsiloxane (PDMS); polyisoprene(PIP); poly(N-hydroxymethyl acrylamide) (PHMA); poly(acrylamide) (PAM); poly(N-hydroxyethyl acrylamide) (PHEA); poly{N-[tris(hydroxymethyl) methyl]acrylamide} (PTHMAM); poly(methacrylamide) (PMA); poly(N-(2-hydroxypropyl)methacrylamide) (PHPMA); poly(vinyl pyrrolidone) (PVP); poly(ethylene oxide) (PEO); latex; titanium dioxide (TiO2), titanium or silicon dioxide (SiO2). The substrate may be PP, PU, PE, uPVC, pPVC, PI, EVA, or PTFE. The substrate may be TiO₂ or SiO₂. The substrate may forms part of an apparatus. The apparatus may be selected from: a urinary device; a dental fixture; an artificial joint; a vascular device; a storage device; blood storage device; a microfluidic device; a filtration membrane; a feed tube; or a diagnostic device. The vascular device may be a catheter, a lead, or a stent. The urinary device may be a urine storage device, blood storage device, catheter, or a stent. The filtration membrane may be a blood filtration membrane, a water purification membrane, or an air purification membrane. The coated substrate may reduce biofouling. The coated substrate may reduce adhesion. The PDMA polymer may be hPDMA or uhPDMA. The PDMA co-polymer may be a copolymer of N,N-Dimethylacrylamide and N-(3-Aminopropyl) Methacrylamide with: an Iodoacetyl Linker (PDMA-co-APMA-I); a Bromoacetyl Linker (PDMA-co-APMA-Br); a Maleimide Linker (PDMA-co-APMA-M); or Pyridyl disulfide Linker (PDMA-co-APMA-Pd). The AMP may be selected from one or more of: E6; Tet20C; Tet20LC; DJK5C; DJK5; DJK6; RI-DJK5; IDR-1018; and 3002C. The AMP may be conjugated by an amine group or a thiol group to a quinone group on the PDA. The AMP may also conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer. The PDMA co-polymer may be a copolymer of N,N-Dimethylacrylamide and N-(3-Aminopropyl) Methacrylamide with: an Iodoacetyl Linker (PDMA-co-APMA-I); a Bromoacetyl Linker (PDMA-co-APMA-Br); or a Maleimide Linker (PDMA-co-APMA-M). The PDMA co-polymer may be a copolymer of N,N-Dimethylacrylamide and N-(3-Aminopropyl) Methacrylamide with: an Iodoacetyl Linker (PDMA-co-APMA-I); or a Bromoacetyl Linker (PDMA-co-APMA-Br).

The hydrophilic polymer may be selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate). The hydrophilic polymer may be selected from PMPC and PMPDSAH. The hydrophilic polymer co-APMA polymer may be selected from PMPC-co-APMA-I and PMPDSAH-co-APMA-I.

The thiol containing hydrophilic molecule may be selected from: 1-thioglycerol; thioethanol; 2-mercaptoethanol; 3-mercapto-1,2-propandiol; and dimercaptosuccinic acid. The bringing the substrate into contact with the PDA and PDMA polymer or a PDMA co-APMA polymer solution, may be by immersing the substrate in the PDA and PDMA polymer or a PDMA co-APMA polymer solution. The bringing the substrate into contact with the AMP solution may be by immersing the substrate in the AMP solution. The immersing of the substrate in the AMP solution may be for between 2-12 hours. The addition of the thiol containing hydrophilic compound to the AMP solution, the substrate may remain in the AMP solution with the thiol containing hydrophilic compound for between 12-24 hours. The addition of the thiol containing hydrophilic compound to the AMP solution, the substrate may remain in the AMP solution with the thiol containing hydrophilic compound for between 20-24 hours. The addition of the thiol containing hydrophilic compound to the AMP solution, the substrate may remain in the AMP solution with the thiol containing hydrophilic compound for between 20-30 hours. The rinsing in (b) and (e) may be with water. The drying in (b) and (e) may be under a stream of argon gas or a flow of nitrogen gas. The drying in (b) and (e) may be under a stream of argon gas. The AMP may be conjugated by an amine group or a thiol group to a quinone group on the PDA.

The polymeric binder may be selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin. The thiol containing hydrophilic molecule may be selected from: 1-thioglycerol; thioethanol; 2-mercaptoethanol; 3-mercapto-1,2-propandiol; and dimercaptosuccinic acid.

In a further embodiment, there is provided a composition comprising AMPs conjugated via amine or thiol groups to quinone groups of PDA in PDA/PDMA coating (BA coating).

In a further embodiment, there is provided a method comprising conjugation of AMPs directly onto PDA component in a non-fouling background generated by PDA/PDMA coating (BA coating) said method comprising a reaction between quinone groups in the underlying PDA and amine or thiol groups in the AMP.

In a further embodiment, there is provided a method comprising conjugation of AMPs via a copolymer approach (MA coating) said method comprising tethering the AMP into the coating between the reaction of iodoacetamide groups on the polymers and thiol groups on the AMP and the reaction between PDA and AMP.

In a further embodiment, there is provided an AMP modified coating structure with optimal protection against non-specific adhesion of dead/live bacteria and the bactericidal and anti-biofilm activity of conjugated AMPs.

In a further embodiment, there is provided an AMP modified coating structure (with cysteine at the C-terminus) with anti-biofilm have potent activity against bacteria including but not limited to S. saprophyticus and E. coli.

In a further embodiment, there is provided a conjugated AMP, Tet20LC, showed the best anti-biofilm activity against S. aureus.

In a further embodiment, there is provided a conjugated AMPs including E6, Tet20C, Tet20LC and DJK5C showed better broad-spectrum activity against different pathogens.

In a further embodiment, there is provided a conjugated AMP, DJK5C, in the coating showed better prevention of biofilm formation than N-terminal conjugated DJK5 for all four bacterial strains tested.

In a further embodiment, there is provided a surface conjugated with AMPs applied to the coating of polymeric surfaces including.

In a further embodiment, there is provided a combination of antimicrobial peptides conjugated to an antifouling coating.

In a further embodiment, there is provided a PDA/PDMA coating (BA coating) with AMP that has optimal protection against non-specific adhesion of dead/live bacteria and retained the bactericidal activity by an electrostatic membrane disruption mechanism.

In a further embodiment, there is provided peptide compositions comprising DJK5C and 3002C.

In a further embodiment, there is provided a peptide compositions comprising the amino acid sequence vqwrairvrvirc (SEQ ID NO:4).

In a further embodiment, there is provided a peptide compositions comprising the amino acid sequence ILVRWIRWRIQWC (SEQ ID NO:7).

The substrate may be a plastic, a rubber, a resin, a metal, a ceramic, a carbon based material, a metal oxide, a hydrogels, a biological tissue, a wood or a cement. The substrate may be poly(propylene) (PP); poly(urethane) (PU); poly(ethylene) (PE); unplasticized polyvinyl chloride (uPVC); plasticized polyvinyl chloride (pPVC); poly(imide) (PI); ethylene vinyl acetate (EVA); poly(tetrafluoroethylene) (PTFE); poly(N-hydroxymethyl acrylamide) (PHMA); poly(acrylamide) (PAM); poly(N-hydroxyethyl acrylamide) (PHEA); poly{N-[tris(hydroxymethyl) methyl]acrylamide} (PTHMAM); poly(methacrylamide) (PMA); poly(N-(2-hydroxypropyl)methacrylamide) (PHPMA); poly(vinyl pyrrolidone) (PVP); poly(ethylene oxide) (PEO); titanium dioxide (TiO₂), titanium or silicon dioxide (SiO₂). The substrate may be poly(propylene) (PP); poly(urethane) (PU); poly(ethylene) (PE); unplasticized polyvinyl chloride (uPVC); plasticized polyvinyl chloride (pPVC); poly(imide) (PI); ethylene vinyl acetate (EVA); poly(tetrafluoroethylene) (PTFE); titanium dioxide (TiO₂) or silicon dioxide (SiO₂). The substrate may be PP, PU, PE, uPVC, pPVC, PI, EVA, PTFE, PHMA, PAM, PHEA, PTHMAM, PMA, PHPMA, PVP, or PEO. The substrate may be TiO₂ or SiO₂. The substrate may form part of an apparatus. The apparatus may be selected from: a urinary device; a dental fixture; an artificial joint; a vascular device; a storage device; a microfluidic device; a filtration membrane; a feed tube; or a diagnostic device or a blood storage device. The vascular device may a catheter, a lead, guide wire, sheath or a stent. The vascular device may a catheter, a lead or a stent. The urinary device maybe a urine storage device, catheter, or a stent. The filtration membrane may be a blood filtration membrane, a water purification membrane, or an air purification membrane.

The methods described herein may be for preventing: biofouling; biofilm formation; protein adsorption; protein binding; cell adhesion; cell growth; microorganism adhesion; and microorganism adhesion and growth. The methods described herein may be for preventing: biofouling; biofilm formation; protein adsorption; protein binding; cell adhesion; microorganism adhesion; and microorganism adhesion and growth. The microorganism may be bacteria. The bacteria may be Gram-positive or Gram-negative bacteria. The gram-positive bacteria may be Staphylococcus aureus (S. aureus). The gram-negative bacteria may be Escherichia coli (E. col). The microorganism may be selected from one or more of the following: E. facium, S. aureus, K. pneumonia, A. baumannii, P. aeruginosa, E. cloacae, E. coli, S. epidermidis, and S. saprophyticus.

### BRIEF DESCRIPTION OF THE DRAWINGS

FIG. 1 shows a schematic representation of conjugation of AMPs onto non-fouling coating with different structure of polymer chains on the surface. (A) Conjugation of AMPs directly onto PDA component in a non-fouling background generated by PDA/PDMA coating (BA coating). The AMPs were conjugated by the reaction between quinone groups in the underlying PDA and amine or thiol groups in the AMP. (B) Conjugation of AMPs via a copolymer approach (MA coating). The AMPs was tethered into the coating between the reaction of iodoacetamide groups on the polymers and thiol groups on the AMP as well as the reaction between PDA and AMP.

FIG. 2 shows a surface characterization of the coatings. (A) ATR-FTIR spectra of BA coating on 4G catheter before and after peptide conjugation and the spectral subtraction. (B) ATR-FTIR spectra of MA coating on 4G catheter before and after peptide conjugation. (C) XPS survey scan of BA and MA coating on silicon wafer before and after peptide conjugation. The change in water angle (D) and thickness (E) of the coating on Ti substrate before and after peptide conjugation. (F) Coating stability. The unnoticeable change in thickness of the coating indicates that it is stable in PBS for 7 days or it can withstand ultrasonication for 10 min. (F) QCM-D real-time analysis of the conjugation of E6 to the BA-coated surface in buffer (pH 8.0)

FIG. 3 shows a surface characterization via atomic force microscopy. Surface morphology of BA (A) and BA-E6 (B) coating on Silicon wafer. AFM force spectroscopy of (C) BA coating and BA-E6 coating on silicon wafer. Representative approach (black line) and retraction (red line) force curves are shown. Distribution of the (D) rupture distance and (E) the corresponding adhesive force for the BA-E6 coated silicon wafer.

FIG. 4 shows a surface characterization via atomic force microscopy. Surface morphology of MA (A) and MA-E6 (B) coating on Silicon wafer. AFM force spectroscopy of (C) MA coating and MA-E6 coating on silicon wafer. Representative approach (black line) and retraction (red line) force curves are shown. Distribution of (D) rupture distance and (E) adhesive force for the MA coating on silicon wafer. Distribution of the (F) rupture distance and (G) adhesive force for the MA-E6 coating on silicon wafer.

FIG. 5 shows the efficiency of coatings in prevention of early-stage biofilm formation. Ti coated silicon wafers were used for the study. Representative fluorescence microscopy images of bacteria adhesion (S. saprophyticus) on (A) uncoated Ti, BA, BA-E6, MA and MA-E6 coatings after 6 h and 24 h incubation in TSB medium. Number and dead percentage of bacteria adhered onto the bare Ti substrate and on different coatings at 6 h (B and C) and 24 h (D and E). Scale bar=100 μm. The morphology of bacteria adhered on the (F) uncoated Ti, (G) BA, (H) BA-E6 and (I) MA-E6 coating. Scale bar=2 μm. Bacterial culture (1 mL) with initial count of ˜5×10⁵ CFU/mL was added on to each sample in a 24 wells plate and incubated at 37° C. on a platform rocking at 50 rpm. Student's two-tailed unpaired t-test was used for statistical analysis (N=3). * indicates p≤0.05, ** indicates p≤0.01, and *** indicates p≤0.001.

FIG. 6 shows the efficiency in prevention of biofilm formation in vitro by different coatings. Representative confocal fluorescence microscopy images of S. saprophyticus biofilm formation on (A) uncoated Ti, (B) BA coating, (C) BA-E6 coating and (D) MA-E6 coating. Representative confocal fluorescence microscopy images of P. aeruginosa biofilm formation on (E) uncoated Ti, (F) BA coating, (G) BA-E6 coating and (H) MA-E6 coating. Scale bar=100 μm. 1 mL bacterial culture with initial count of ˜5×10⁵ CFU/mL was added to each sample in the wells of 24 wells plate and incubated at 37° C. on a platform rocking at 50 rpm for 24 h.

FIG. 7 shows the efficiency in prevention of biofilm formation in vitro. (A) The reduction of bacterial adhesion (S. saprophyticus) on coated 14G PU catheter after incubation in TSB medium for 24 h. (B) The reduction of bacterial adhesion (P. aeruginosa) on coated 14G PU catheter after incubation in LB medium for 24 h.

FIG. 8 shows a semi-throughput screening method for the identification of high efficiency anti-biofilm surface immobilized peptides in relevant non-fouling background. (A) Schematic of BA-AMP coating on 96 wells plate. Different from the conventional tethered AMPs on the substrate, the AMPs in the BA-AMP coating is presented in a non-fouling background generated by PDMA chains. (B) Increase in thickness after peptide conjugation on the BA coating modified silicon substrate. Screening of tethered peptides utilizing BA-AMP coating against S. saprophyticus (C), S. aureus (D), P. aeruginosa (E) and E. coli (F). ns: not significant difference. *.p<0.05; **p<0.01; ***p<0.001.

FIG. 9 shows hemolysis and cytocompatibility of BA and BA-E6 coating on PU surface. (A) Percentage of hemolysis brought by the BA and BA-E6 coated PU coupon (diameter of ⅝″), (B) Viability of T24 cells grown on the BA and BA-E6 coated PU coupon.

FIG. 10 shows inhibition of biofilm formation by S. saprophyticus by the BA coating conjugated with E6 or Tet20LC on PU catheters in vitro and in mouse urinary tract infection model. (A) Number of survived S. saprophyticus recovered from the 24G PU catheter surface after incubating for 24 h in vitro. (B) Number of S. saprophyticus adhered on the PU catheter surface and (C) in the urine after 7 days in vivo in urinary infection model. SEM images of biofilm on (D) bare and (E) BA coating conjugated with E6, (F) Tet20LC coated catheter after instillation in mice for 7 days. (Scale bar=5 μm).

FIG. 11 shows ¹HNMR spectra of synthesized copolymer PDMA-co-APMA

FIG. 12 shows the ¹HNMR spectra of modified copolymer PDMA-co-APMA-I

FIG. 13 shows AFM force spectroscopy of polydopamine conjugated with E6 peptide (PDA-E6) coated Silicon wafer, representative approach (red line) and retract (blue line) force curves are shown.

FIG. 14 shows SEM images of biofilm of S. saprophyticus on (A) bare catheter, (B) BA-E6 coated catheter, and (C) BA-Kai88 coated catheter after instillation of S. saprophyticus in vivo for 7 days. (scale bar=100 μm).

FIG. 15. Screening of tethered peptides utilizing BA-AMP coating against S. saprophyticus (A, B) and S. epidermidis (C). OD reading (A) and planktonic concentration (B) of S. saprophyticus after 24 h culture in uncoated and coated wells. Planktonic concentration (C) of S. epidermidis after 24 h culture in uncoated and coated wells. *p<0.05; **p<0.01; ***p<0.001.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

Any terms not directly defined herein shall be understood to have the meanings commonly associated with them as understood within the art of the invention.

The term “high molecular weight polymer” or hMWP as used herein refers to any polymer having a molecular weight between ≥100,000 daltons (i.e. greater than and equal to 100 kDa) and about ≤200 kDa and in particular refers to the hydrophilic polymers described herein, including the hydrophilic biocompatible polymer poly(N,N-dimethyl acrylamide) (PDMA). Alternatively, the HMW polymer may be selected on the basis of having a polydispersity index (PDI) of between 1 to 3.

The term “ultra-high molecular weight polymer” or uhMWP as used herein refers to any polymer having a molecular weight >200 kDa and in particular refers to the hydrophilic polymers described herein.

The present disclosure provides, in part, AMPs conjugated to polymer coating surfaces. The conjugated AMPs have broad spectrum activity against biofilms.

The polymer-AMP coatings can be deposited on diverse biomedical material surfaces.

The contact killing by the AMP, and repulsion or release of live/dead bacteria from the surface by the non-fouling component prevents biofilm formation.

The present disclosure provides, in part, a simple universal antifouling coating approach which has the ability to perform with potent surface activity and can also easily translated as a biomedical device coating

When the peptide was presented within the antifouling layer without direct attachment to the polymer chains (BA-AMP coating) potent long-term biofilm activity is achieved due to the AMPs being sterically protected.

The BA-AMP coating structure offered optimal protection of AMPs against non-specific adhesion of dead/live bacteria while retaining the bactericidal and anti-biofilm activity of conjugated AMPs. The PDMA chains in the moderate brush regime were responsible for the antifouling characteristics of the BA-AMP coating and was helpful in preventing the accumulation of bacterial debris on the surface, the current coating approach is simple, and versatile, and can be applied to diverse materials used in medical device manufacturing, making its translation potential into clinical practice high.

A “polymeric binder” as used herein may be selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin. A polymeric binder may also be selected from catechol and catechol derivative polymers as well known in the art^{[36]}.

The term “peptide” as used herein includes any structure comprised of two or more amino acids, including chemical modifications and derivatives of amino acids. The amino acids forming all or a part of a peptide may be naturally occurring amino acids, stereoisomers and modifications of such amino acids, non-protein amino acids, post-translationally modified amino acids, enzymatically modified amino acids, constructs or structures designed to mimic amino acids, and the like, so that the term “peptide” includes pseudopeptides and peptidomimetics, including structures which have a non-peptidic backbone. The amino acids in a “peptide” may be in either the D or the L-configuration. Furthermore, “peptide” is meant to include dimers or multimers and peptides produced by chemical synthesis, recombinant DNA technology, biochemical, or enzymatic fragmentation of larger molecules, combinations of the foregoing or, in general, made by any other method. Peptides as used herein, optionally with non-amino acid residue groups at the N- and C-termini, such groups including acyl, acetyl, alkenyl, alkyl, N-alkyl, amine, or amide groups, among others, as described in more detail below in reference to “peptide modifications”.

The term “antimicrobial peptide” (AMP) refers to diverse group of peptide molecules, which are generally between 10 and 50 amino acids, but may extend to over a hundred amino acids. These peptides are potent, broad spectrum antibiotics which demonstrate potential as novel therapeutic agents. Some AMPs are effective against Gram-positive bacteria, Gram-negative, fungi and some viruses. AMPs are divided into subgroups on the basis of their amino acid composition and structure (i.e. anionic peptides; linear cationic α-helical peptides; cationic peptide enriched for specific amino acid; and anionic/cationic peptides forming disulfide bonds). Furthermore, AMPs are regularly used as therapeutic agents (for example, Bacitracin; Boceprevir, Dalbavancin; Daptomycin; Enfuvirtide; Oritavancin; Teicoplanin; Telaprevir, Telavancin; Vancomycin; and Guavanin 2). Non-limiting examples of AMPs as described herein are as follows: E6; Tet20C; Tet20LC; DJK5C; DJK5; DJK6; RI-DJK5; IDR-1018; and 3002C. The amino acids in the AMPs may be in either the D or the L-configuration. The AMPs may include those sequences identified in TABLE 3. However, many such peptides are known in the art^{[53, 54, 55]} and would be suitable for use with the present compositions and coatings.

As used herein “a linking moiety” is used to attach the hydrophilic polymer co-polymer with the AMP. This may be accomplished via numerous linking groups, for example, by adding: an alkyne group (via copper assisted or copper free click reactions or a peptide with azide functionality can be used); an alkene group (thiolene click reaction, where a peptide with an —SH group can be attached); an aldehyde group (via Schiff-based reactions of aldehyde group on surface and —NH2 group on peptides); or via enzymatic ligation of peptides to the surface (e.g. transglutaminase-reaction of an AMP with glutamine with amine residues on the surface). Furthermore, the linker may be selected from one or more of the following: an iodoacetyl linker (L=CH₂I); a bromoacetyl linker (L=CH₂Br); a Maleimide Linker (i.e. L=

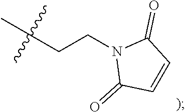 [SMILES: *CCN1C(=O)C=CC1=O]

and Pyridyl disulfide Linker (L=

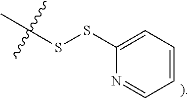 [SMILES: *SSc1ccccn1]

The term “peptide modifications” as used herein refers to any modification to a peptide improves the characteristics of the peptide to act as a bound anti-microbial peptide (AMP). For example, modifications may reduce susceptibility to proteolysis, improve binding affinities, and/or confer or modify other physicochemical or functional properties. Examples of modifications include but are not limited to phosphorylation; acetylation; N-linked glycosylation; amidation; hydroxylation; methylation; O-linked glycosylation; ubiquitylation; pyrrolidone carboxylic acid; and sulfation. Alternative peptides modifications may include: single or multiple amino acid substitutions (e.g., equivalent, conservative or non-conservative substitutions, deletions or additions) may be made in a sequence; the peptide or peptide analog is lipidated (e.g., myritoylated, palmitoylated, or other linking to a lipid moiety), glycosylated, amidated, carboxylated, phosphorylated, esterified, acylated, acetylated, cyclized, pegylated to or converted into an acid addition salt and/or optionally dimerized or polymerized, or conjugated.

One type of peptide modification is C-terminal amidation. C-terminal amidation removes the charge form the C-terminus of a peptide and may reduce the overall solubility of the peptide. Having an uncharged C-terminal amide end more closely mimics the native protein, and therefore may increase the biological activity of a peptide. Alternatives include N-terminal acetylation, which may increase peptide stability by preventing N-terminal degradation.

The term “biofilm” is used herein as it is normally understood to a person of ordinary skill in the art and often refers to any group of organisms adhering to the surface of a structure.

The term “biofouling” is used herein as it is normally understood to a person of ordinary skill in the art and often refers to the colonization of an interface by organisms, which often leads to deterioration of the interface.

The term “antifouling” is used herein as it is normally understood to a person of ordinary skill in the art and often refers to the reduction of formation of biofilms and biofouling.

The term “plastic” as used herein is meant to encompass a vast number of synthetic or semi-synthetic organic polymers that are malleable and may be molded into solid forms. Exemplary plastics are: Polyester (PES); Polyethylene terephthalate (PET); Polyethylene (PE); High-density polyethylene (HDPE); Polyvinyl chloride (PVC); Polyvinylidene chloride (PVDC); Low-density polyethylene (LDPE); Polypropylene (PP); Polystyrene (PS); High impact polystyrene (HIPS); Polyamides (PA) (Nylons); Acrylonitrile butadiene styrene (ABS); Polyethylene/Acrylonitrile Butadiene Styrene (PE/ABS a blend of PE and ABS); Polycarbonate (PC); Polycarbonate/Acrylonitrile Butadiene Styrene (PC/ABS a blend of PC and ABS); Polyurethane (PU); Polylactic acid (PIA); Polyimide; Polyetherimide (PEI); Polyetheretherketone (PEEK); phenol formaldehydes (PF); and Polymethyl methacrylate (PMMA).

The term “polydopamine (PDA)” is used herein as it is normally understood to a person of ordinary skill in the art and often refers to the pH-dependent self-polymerization of dopamine. However, “polydopamine” may be formed by any polymerisation of dopamine monomers. It should be noted that the mechanism of PDA formation is currently not understood^{[37-38]}. Furthermore, it should be noted that the structure of the polymer product has not been elucidated yet^{[37]}.

The term “PDMA” is used herein as it is normally understood to a person of ordinary skill in the art and often refers to poly(N,N-dimethyl acrylamide).

The term “PMPDSAH” is used herein as it is normally understood to a person of ordinary skill in the art and often refers to “poly(N-(3-(methacryloylamino)propyl)-N,N-dimethyl-N-(3-sulfopropyl) ammonium hydroxide)”.

The term “PMPC” is used herein as it is normally understood to a person of ordinary skill in the art and often refers to “poly(2-methacryloyloxyethyl phosphorylcholine)”.

The term “PP” is used herein as it is normally understood to a person of ordinary skill in the art and often refers to “poly(propylene)”.

The term “PU” is used herein as it is normally understood to a person of ordinary skill in the art and often refers to “poly(urethane)”.

The term “PE” is used herein as it is normally understood to a person of ordinary skill in the art and often refers to “poly(ethylene)”.

The term “uPVC” is used herein as it is normally understood to a person of ordinary skill in the art and often refers to “unplasticized polyvinyl chloride”.

The term “pPVC” is used herein as it is normally understood to a person of ordinary skill in the art and often refers to “plasticized polyvinyl chloride”.

The term “PI” is used herein as it is normally understood to a person of ordinary skill in the art and often refers to “poly(imide)”.

The term “EVA” is used herein as it is normally understood to a person of ordinary skill in the art and often refers to “ethylene vinyl acetate”.

The term “Teflon” is used herein as it is normally understood to a person of ordinary skill in the art and often refers to “poly(tetrafluoroethylene) or PTFE”.

The term “PHMA” is used herein as it is normally understood to a person of ordinary skill in the art and often refers to “poly(N-hydroxymethyl acrylamide)”.

The term “PAM” is used herein as it is normally understood to a person of ordinary skill in the art and often refers to poly(acrylamide).

The term “PHEA” is used herein as it is normally understood to a person of ordinary skill in the art and often refers to “poly(N-hydroxyethyl acrylamide)”.

The term “PTHMAM” is used herein as it is normally understood to a person of ordinary skill in the art and often refers to “poly{N-[tris(hydroxymethyl) methyl]acrylamide}”.

The term “PMA” is used herein as it is normally understood to a person of ordinary skill in the art and often refers to “poly(methacrylamide)”.

The term “PHPMA” is used herein as it is normally understood to a person of ordinary skill in the art and often refers to “poly(N-(2-hydroxypropyl)methacrylamide)”.

The term “PVP” is used herein as it is normally understood to a person of ordinary skill in the art and often refers to “poly(vinyl pyrrolidone)”.

The term “PEO” is used herein as it is normally understood to a person of ordinary skill in the art and often refers to “poly(ethylene oxide)”.

A hydrophilic co-polymer may have the structure of Formula I

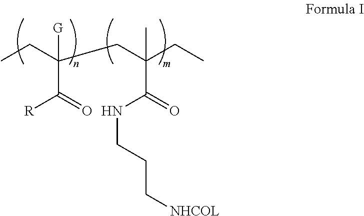

wherein,
G is H or —CH₃;
L is a linking moiety; and
R is selected from

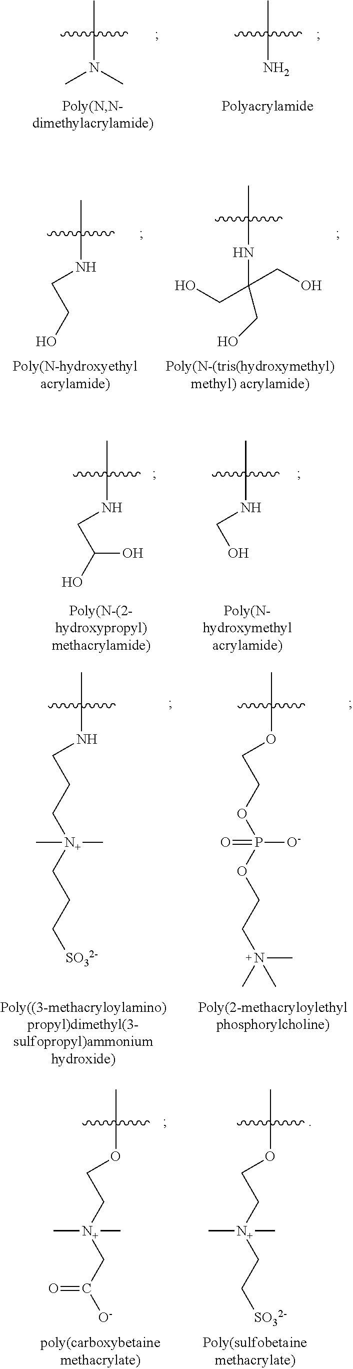

L may be selected from: CH₂I; CH₂Br;

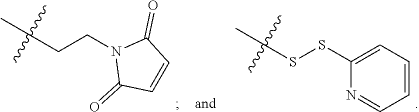 [SMILES: *CCN1C(=O)C=CC1=O ; *SSc1ccccn1]

The term “coating” is used herein as it is normally understood to a person of ordinary skill in the art to be a covering that is applied to the surface of an object and is to be broadly constructed to include adhesive coating, resistive coating (e.g., resistive to cellular adhesion), and protective coating. The present invention offers adhesion in “highly humid” environments (50% to 80% humidity) and “wet”, “saturated”, or “super-saturated” environments (at least 80% humidity and higher). Adhesion under dry environment is also contemplated herein.

The term “dip-coating” is used herein as it is normally understood to a person of ordinary skill in the art and often refers to the immersion of the substrate into the solution of the coating material.

The term “lubricity” is used herein as it is normally understood to a person of ordinary skill in the art and often refers to the property of “slipperiness” or “smoothness”, or “a surface with low friction”.

The coating described herein has high lubricity. These coatings are useful for medical devices where their lubrication results in reduced frictional forces when the device is introduced and moved within the body, reducing inflammation and tissue trauma as well as supporting patient comfort.

Various alternative embodiments and examples are described herein. These embodiments and examples are illustrative and should not be construed as limiting the scope of the invention.

### MATERIALS AND METHODS

### Materials

Ultra-high molecular weight poly(N,N-dimethylacrylamide) (uhPDMA) and high molecular weight poly(N,N-dimethylacrylamide) (hPDMA) were synthesized by atom transfer radical polymerization 391. N-(3-Aminopropyl) methacrylamide hydrochloride (APMA) (98%) was purchased from Polysciences™, USA and was used as supplied. All other reagents including 1, 1, 4, 7, 10, 10-hexamethyl triethylene tetramine (HMTETA) (97%), Tris[2-(dimethylamino)ethyl]amine (Me₆TREN) 97%, methyl 2-chloropropionate (97%), CuCl (99%), CuCl₂ (99%), 1-thioglycerol (97%) were purchased from Sigma-Aldrich™ (Oakville, ON). A single-side-polished silicon wafer (University Wafer™, Boston, MA) deposited with titanium was prepared by e-beam evaporation of titanium. The process was progressed in a home-assembled Evaporator 2000™ system equipped with a quartz crystal microbalance to monitor the film thickness and a cryo pump to reach high-vacuum (10⁻⁷-10⁻⁶ Torr) condition. After deposition, the substrates were washed with Milli-Q water™, dried via a nitrogen gun, and stored for further usage. SurFlash™I.V. Catheter Cat. #SR*FF1451, #SR*FF2419 (14 Gauge and 24 Gauge) made of polyurethane (PU), were purchased from Terumo™. Antimicrobial peptides, E6 (RRWRIVVIRVRRC-NH₂(SEQ ID NO:1)), Tet20C (KRWRIRVRVIRKC-NH₂ (SEQ ID NO:2)), DJK5C (vqwrairvrvirc-NH₂ (SEQ ID NO:4)) and 3002C (ILVRWIRWRIQWC-NH₂ (SEQ ID NO:7)) with cysteine at the C-terminus (purity>95%) were synthesized by Canpeptide Corp.™ (Quebec, Canada). Peptide IDR-1018 (VRLIVAVRIWRR-NH₂ (SEQ ID NO:6)) at >95% purity was obtained from CPC Scientific™ (Sunnyvale, CA), while all other synthetic peptides (>95% purity) were obtained from GenScript™ (Piscataway, NJ). Tet20LC (KRWRIRVRVIRK-bA-bA-C—NH₂(SEQ ID NO:3)) was synthesized and purified (>90%) by the Hilpert laboratory by automated solid-phase peptide synthesis (SPPS) on a MultiPep RSI Peptide Synthesizer (INTAVIS™, Tuebingen, Germany) using the 9-fluorenyl-methoxycarbonyl-tert-butyl (Fmoc/tBu) strategy. Crude peptides were purified to homogeneity of >90% by preparative RP HPLC on a Shimadzu™ LC2020 system equipped with a Jupiter™ 10 μm Proteo C18 column (90 Å, 250×21.2 mm, Phenomenex™) using a linear gradient system containing 0.01% (v/v) TFA in H₂O (solvent A) and 0.01% (v/v) TFA in acetonitrile (solvent B). Pure products were finally characterized by analytical reverse phase high performance liquid chromatography (RP-HPLC) and liquid chromatography-mass spectrometry (LC-MS). The broth microdilution method with minor modifications for cationic peptides was used for measuring the MICs of peptides.

### Synthesis of copolymer of N,N-dimethylacrylamide and N-(3-Aminopropyl) methacrylamide (PDMA-co-APMA)

Copper (II) chloride (CuCl₂, 3 mg), copper (I) chloride (CuCl, 20 mg) and Me₆TREN (120 μL) were added successively into a glass tube followed by the addition of 20 mL Milli-Q water™. The solution was degassed using three freeze-pump-thaw cycles, DMA (2 mL) and APMA (346 mg) was added into the glass tube and degassed with another freeze-pump-thaw cycle. Soluble methyl 2-chloropropionate (20 μL from a stock solution of 40 μL in 5 mL methanol) was added immediately to the reaction mixture, and the polymerization was allowed to proceed at RT (22° C.) for 24 h. The soluble polymer formed was collected and purified by dialysis (molecular weight cut off: 1000 Da) against water (pH was adjusted to 8 by using 0.1M NaOH) for 3 days with daily exchange of water. The polymer was lyophilized to obtain the final product. The absolute molecular weight of PDMA-co-APMA was determined by gel permeation chromatography (GPC) using a DAWN HELEOS II™ multi angle laser light scattering (MALLS) detector (Wyatt Technology Inc.™), an Optilab T-rEX refractive index detector and a quasi-elastic light scattering (QELS) detector (Wyatt Technology Inc.™) in 1.0 M NaNO₃ (pH 7) aqueous solution. NMR spectra were recorded on a Bruker Avance™ 300 MHz NMR spectrometer using deuterated solvents (Cambridge Isotope Laboratories™, 99.8% D) with the solvent peak as a reference.

### Synthesis of PDMA-co-APMA with iodoacetyl linker (PDMA-co-APMA-I)

The copolymer (PDMA-co-APMA, 100 mg) was dissolved in anhydrous acetonitrile (10 mL). Iodoacetic acid N-hydroxysuccinimide ester (70 mg) was added into the solution. The reaction was allowed overnight with stirring. The solution was dialyzed against water for 3 days with daily exchange of water. The solution was finally dialyzed against 5 mM Tris buffer (at pH 8.5) and concentrated to the solid content about 12 mg/mL as measured by Thermogravimetric Analysis (TGA Q500, TA Instruments™, New Castle, DE, USA) and characterized by NMR.

### Preparation of Coating on the Titanium, Polyurethane Catheters and 96 Well Plate Surface Titanium Substrate

Titanium substrates were initially cleaned with nitrogen gas at a flow rate of 50 SCCM (standard cubic centimeter per minute) and at a pressure of 350 mTorr. A plasma power (75 W) was used for 3 min in a M4L Plasma Processing System from PVA Tepla™ (Corona, California, USA). PDMA or PDMA-co-APMA-I solution was prepared at a concentration of 12 mg/mL in 10 mM Tris buffer, pH 8.5, respectively. Dopamine was freshly prepared at a concentration of 12 mg/mL in 10 mM Tris buffer, pH 8.5 before each experiment. The dopamine solution was then mixed with PDMA or PDMA-co-APMA-I solution with a volume ratio of 1:5 (dopamine:polymer) and used right after. The cleaned Ti substrate was dipped into a mixed solution (0.7 mL) of dopamine and polymer in the wells of 24 well-plate for 24 h. The coated substrate was then rinsed by Mill-Q water™ and dried under argon.

### PU Catheter

PU catheter with different size (14G and 24G) were initially cleaned by nitrogen plasma treatment and coated using the same protocol as Ti substrate. For the 24G catheter used for the in vivo study, a second coating (PDA/PDMA) using the same solution composition was then applied onto the catheter to increase the thickness and coverage.

### On Microtiter 96-Well Plate

Microtiter 96-well plates were initially cleaned by nitrogen plasma treatment similar to the Ti substrate. The wells in 96-well plate were then coated with dopamine/PDMA mixed solution (250 μL, 2 mg/ml and 10 mg/ml respectively in 10 mM Tris buffer, pH 8.5). A second coating was applied using 200 μL solution having the same composition. The coated 96-well plate was then rinsed by Mill-Q water™ and dried under a stream of argon gas.

AMP Conjugation onto PDA/PDMA and PDA/PDMA-Co-APMA-I Coated Surfaces

The Ti surface or catheter coated with PDA/PDMA or PDA/PDMA-co-APMA-I was fully immersed into the peptide solution (0.6 mL, 0.1 mg/mL in 10 mM phosphate buffer (pH ˜8) overnight followed by adding 1-thioglycerol (6 μL, at a final concentration of 10 μL/mL) for 24 h. For the 96 well-plate, 250 μL peptide solutions (0.1 mg/mL in 10 mM phosphate buffer, pH ˜8) were added into the wells and incubated overnight followed by addition of 1-thioglycerol (2.5 μL, final concentration of 10 μL/mL) for 24 h. The peptide immobilized substrates were thoroughly rinsed with Milli-Q water™ consecutively and dried under argon. The mass of peptides grafted on to the surface was calculated by the following equation m=ρ·h·A: where h is the increase in thickness after peptide conjugation measured by ellipsometry; ρ is the volumetric mass density of antimicrobial peptide (1.5 mg/cm³);⁴⁸ A is the surface area (cm²).

### Anti-Biofilm Efficiency of AMPs Assembled on Ti Substrate

Pseudomonas aeruginosa (luminescence tagged strain PAO1 Tn7:Plac-lux), and S. saprophyticus strain (ATCC 15305) were sub-cultured for testing the anti-biofilm activity of the coating. An initial concentration 5×10⁵ CFU/mL was used for these analyses. The coated Ti substrate along with the pristine Ti substrate were placed into a 24-well plate and was sterilized by submerging in 1 mL of 70% ethanol for 5 min. Ethanol was then removed, and samples were each rinsed with 1 mL of sterile phosphate-buffered saline (PBS) for a total of 3 times. After the removal of LB media from the last rinse, 1 mL of the prepared bacterial culture (˜5×10⁵ CFU/mL) of S. saprophyticus in TSB or P. aeruginosa in LB was added to each sample. The 24-well plate was incubated at 37° C. with shaking at 50 rpm for 6 h or 24 h. The samples were then thoroughly rinsed with PBS buffer, stained with green SYTO9 for all bacteria and red propidium iodide (red PI) for dead bacteria. The stained bacteria on the Ti surface were examined by fluorescence microscopy (Zeiss Axioskop™ 2 plus, Thornwood, NY) equipped with a fluorescence illumination system (AttoArc™ 2 HBO) and appropriate filter sets. Images were randomly acquired on different spots by using a 10× and 20× objective lens. Images were taken using the filters for fluorescein isothiocyanate and rhodamine to visualize the presence of live and dead bacteria on the surface. The images taken using two different filters were overlaid to generate the merged image by using imageJ v153a at an opacity of 50%. The total number of adhered bacteria was counted using imageJ™. The antimicrobial activity was calculated by dividing the number of dead bacteria by the total number of bacteria. The samples that were incubated for 24 h also analyzed using C2+ confocal microscope (Nikon™) with the 488 and 561 nm channels. All images were acquired using identical acquisition settings. The experiments were repeated 3 times, and representative results from an experiment are shown.

### Anti-Biofilm Efficiency of AMPs Assembled on the Catheter Surface

Catheters with different sizes, 14G and 24G PU, were used in this study. The coated and uncoated catheter samples were cut into 1 cm section and suspended in 70% ethanol for 5 min. The ethanol was removed, and samples were rinsed in sterile PBS for a total of 3 times. After the last rinse, each sample was introduced to culture medium containing approximately 1 mL of 5×10⁵ CFU/mL bacteria (S. saprophyticus in TSB and P. aeruginosa in LB) per sample in Eppendorf tubes. All samples were incubated at 37° C. on a 360° rotator. At 24 h, post-incubation, samples (N=5 per condition) were rinsed with sterile PBS buffer 3 times. Rinsed samples were then transferred to 1 mL of sterile PBS and sonicated for 10 min in a water bath. Samples were vortexed at high speed for 10 s, then serially diluted and plated on LB agar for CFUs. Plates were incubated at 37° C. overnight until visible colonies form. The experiment has been repeated 3 times with similar results, and the representative results from one experiment are shown.

Screening and Identification of Surface Conjugated AMPs with Anti-Biofilm Activity on a 96-Well Plate
Bioluminescence-tagged bacterial strains used in this study included S. aureus (Xen36), P. aeruginosa (PAO1.lux), E. coli (E38.lux), and S. saprophyticus (ATCC 15305). Bacteria were cultured in LB, with shaking at 250 rpm, at 37° C. Bacterial growth was monitored using a spectrophotometer at the optical density of 600 nm (OD₆₀₀). S. aureus Xen36 was purchased from PerkinElmer Inc.™ while P. aeruginosa PAO1.lux and E. coli E38.lux were generated by conjugating in plasmids that constitutively-expressed lux reporter genes^{[40]}. The biofilm inhibitory activity of AMPs conjugated onto coated polypropylene surfaces were evaluated in a static microtitre plate assay as previously described with some modifications^{[41]}. For all experiments, 96-well Costar™ polypropylene plates (Corning™) were used. An overnight culture of selected bacterial strains in LB was diluted to an OD₆₀₀=0.01 in TSB or basal medium 2 (BM2) supplemented with 0.1% or 0.4% glucose and 0.5 mM MgSO₄, (the biofilm growth medium for different bacteria is listed in TABLE 1).

TABLE 1
The six ESKAPE pathogen bioluminescent
strains used in this protocol

| | Overnight | |
|---|---|---|
| | culture | |
| Bacterium | medium | Biofilm growth medium (MBIC) |
| E. facium | Luria-Bertani | 50% TSB (v/v) in sterilized dH₂O, |
| | (LB) | 0.125% glucose (w/v), pH 7.0. |
| S. aureus | Luria-Bertani | 50% TSB (v/v) in sterilized dH₂O, |
| | (LB) | 0.125% glucose (w/v), pH 7.0. |
| K. pneumonia | Luria-Bertani | 1X BM2, 0.4% glucose (w/v), 0.5 mM |
| | (LB) | MgSO₄, pH 7.5. |
| A. baumannii | Luria-Bertani | 1X BM2, 0.4% glucose (w/v), 0.5 mM |
| | (LB) | MgSO₄, pH 7.5. |
| P. aeruginosa | Luria-Bertani | 1X BM2, 0.4% glucose (w/v), 0.5 mM |
| | (LB) | MgSO₄, pH 7.5. |
| E. cloacae | Luria-Bertani | 1X BM2, 0.4% glucose (w/v), 0.5 mM |
| | (LB) | MgSO₄, pH 7.5. |
| E. coli | Luria-Bertani | 1X BM2, 0.4% glucose (w/v), 0.5 mM |
| | (LB) | MgSO₄, pH 7.5. |
| S. epidermidis | Luria-Bertani | 50% TSB (v/v) in sterilized dH₂O, |
| | (LB) | 0.125% glucose (w/v), pH 7.0. |
| S. | Luria-Bertani | 100% TSB (v/v) in sterilized dH₂O, |
| saprophyticus | (LB) | 1% glucose (w/v), pH 7.0. |

For inhibition experiments, 100 μL of the diluted overnight culture was added to each well in the plate coated with control coating and AMP coating, respectively. After overnight growth under static condition at 37° C., planktonic cells were removed, adhered biomass rinsed three times with distilled water and subsequently the remaining adhered total biomass was quantified by measuring the luminescence (Total white light) using the Synergy H1™ multimode microtitre plate reader (BioTek™). The remaining adhered S. saprophyticus biomass was quantified using the crystal violet (CV) staining. Briefly, each well was added and incubated with 125 μL of 0.1% (v/v) CV solution for 30 minutes at room temperature with moderate shaking. Then, CV was discarded, and each well was rinsed three times with distilled water. The remaining CV stain was resuspended in 150 μL of 70% ethanol for 30 minutes at room temperature with moderate shaking and then quantified by measuring OD₅₉, using the Synergy H1™ multimode microplate plate reader (BioTek™). The percent biofilm inhibition was calculated in relation to the amount of biofilm grown in the absence of the coating (defined as 100%) and the media sterility control (defined as 0% growth). The experiment was repeated 3 times with 3 technical replicates per biological replicate.

### Evaluation of Anti-Biofilm Efficiency of AMP Coating on 24G PU Catheters in a Mouse Urinary Infection Model

A total of 48 male C57BL/6 mice (Harlan™) at 10 weeks of age were included in experiments. Twenty mice were included in the control group (bare catheter) and 14 mice for each treated group (AMPs conjugated catheter, E6 and Tet20LC). The implantation of the catheter followed the procedure published previously 421. Briefly, prior to animal procedures, 4 mm section from the tip of PU catheter was cut using sterile blades and re-assembled back onto the original needle. The assembled catheter was sterilized using ethylene oxide. All mice were anesthetized using 3% isoflurane. The abdominal area was shaved, and the area around the mouse bladder was secured. Sterile ultrasound gel was applied to visualize the bladder. The 24G PU catheter assembly was positioned at a 30-degree angle just above the pubic bone with the bevel directed to the anterior. The catheter assembly was carefully inserted towards the bladder and left the 4 mm catheter segment inside while the ‘pusher’ was pushed slightly inward. One day after catheter implantation, all mice were anaesthetized and S. saprophyticus (1×10⁷ CFU/mL in 50 μL PBS) was percutaneously injected into the bladder. Mice were kept anaesthetized with 1% isoflurane for 1 h on a heating pad to allow time for bacteria to adhere onto the implanted catheter. At 7 days post-instillation of S. saprophyticus, all mice were sacrificed by CO₂ asphyxiation. The presentation of urine in the bladder was examined by ultrasound. If present, urine samples were collected from the bladder. In the case of urine not presenting in the bladder, 50 μL PBS buffer was injected into the bladder to rinse the bladder wall. The number of bacteria in the urine was quantified via serial dilutions and CFU counts. Indwelling catheters were collected, rinsed in 200 μL of sterile PBS three times and finally placed in 100 μL PBS. Seventeen from 20 explanted catheter samples in the control group and eleven from 14 for the coated group were sonicated for 10 minutes to aid biofilm dispersal. Samples were then vortexed at high speed for 10 sec, and bacterial numbers were determined by serial dilutions and CFU counts. Three catheters from each group were fixed in 2.5% glutaraldehyde (200 μL) for 1 h, and then dehydrated using an ethanol/water gradient (50%, 70%, 90%, 100%) for 10 min each. The samples were dried in the ambient condition, sputtered with Au/Pd (˜10 nm) and viewed using a scanning electron microscope (SEM, Hitachi™ SU3500) to visualize biofilm formation on catheter surface. There were 5 cases of unsuccessful infection with S. saprophyticus in the control group and 4 in the coated group of E6 and 2 in the coated group with Tet20LC as the CFU reading on both catheter surface and in urine was zero.

### Surface Characterization of AMP Coating

ATR-FTIR analysis: Absorption spectra of different surface coatings (both control and AMP conjugated) on catheter surface were collected on a Bruker 670 TensorII™ with a MCT/A liquid nitrogen cooled detector, a KBr beam splitter, and a VariGATR™ Grazing Angle accessory. Spectra were recorded at 4 cm⁻¹ resolution, and 128 scans were collected. Water contact angle measurements: A water droplet (6 uL) was placed on the surface and an image of the droplet was taken with a digital camera (Retiga 1300™, Q-imaging Co.™). The contact angle was analyzed using Northern Eclipse™ software. Over three different sites were tested for each sample. X-ray photoelectron spectroscopy(XPS): Measurements were carried out at Nanofabrication and Characterization Facility (nanoFAB™), University of Alberta. The spectra were collected using a Kratos Axis Ultra™ X-ray photoelectron spectrometer operated in energy spectrum mode at 210 W Spectra were fit using CasaXPS (VAMAS) software and were calibrated to the lowest binding energy component of the Cis emission at 284.6 eV. Ellipsometry measurements: The variable-angle spectroscopic ellipsometry (VASE) spectra were collected on an M-2000 V spectroscopic ellipsometer (J. A. Woollam, Lincoln, NE) at 50°, 60°, and 70° at wavelengths from 480 to 700 nm with an M-2000 50W quartz tungsten halogen light source. The VASE spectra were then fitted with a multilayer model utilizing WVASE32 analysis software based on the optical properties of a generalized Cauchy layer to obtain the dry thickness of the deposited layers. OCM measurements: AMP immobilization on the surfaces were quantitatively monitored in real-time by QCM-D™ (Q-sense AB™, Sweden) at room temperature. Briefly, the BA coating was deposited on SiO₂ (˜50 nm) coated sensors. For the evaluation of AMP E6 binding to the BA functionalized surface, the sensors were mounted into the QCM-D chamber. After stabilization of the baseline with buffer, a 0.1 mg/mL AMP E6 solution (pH 8.0) was flowed (50 μL/min) over the sensor. Finally, phosphate buffered saline (pH 8.0, 10 mM) was perfused to remove physically absorbed peptides. The measurements of the resonant frequency were carried out continuously. The mass was fitted through the Sauerbrey equation by using Qsense Dfind 1.2.2. Atomic force microscopy analysis: Measurements were performed on a commercially available multimode system (Dimension 3100™) with an atomic head of 130×130 μm² scan range which used a NanoScope IIIa™ controller (Digital Instruments™, Santa Barbara, CA). The surface morphology and adhesive force in PBS buffer was collected in the contact mode using commercially manufactured V-shaped silicon probe (Bruker™, NP—S10) with a spring constant ˜0.06 N/m.

### Applying PDMA/PDA (BA) Coating on the 96-Well Polypropylene Plate

The uhPDMA was prepared at a concentration of 36 mg mL⁻¹ in sodium acetate buffer (50 mM, pH=5). Dopamine was freshly prepared at a concentration of 12 mg mL⁻¹ in sodium acetate buffer (50 mM, pH=5) before each experiment. Sodium periodate (NaIO₄) was freshly prepared at a concentration of 28 mg mL-1 in sodium acetate buffer (50 mm, pH=5). The dopamine solution was then mixed with uhPDMA solution with a volume ratio of 1:5 (dopamine:uhPDMA). Sodium periodate solution was then added to generate a final concentration of uhPDMA at 30 mg mL⁻¹, dopamine at 2 mg mL⁻¹, and NaIO₄ at 0.9 mg mL⁻¹. Mixed solution (200 μL) was added into the wells of 96-well polypropylene plate. The plate was placed onto a rocking platform with shaking at 50 rpm for 2 hr. The coated plate was then rinsed by Milli-Q™ water and dried for further characterization.

Antimicrobial Peptide (AMP) Conjugation onto the PDMA/PDA Coated 96-Well Polypropylene Plate

Peptide solutions (200 μL, 0.25 mg/mL in 10 mM phosphate buffer, pH ˜8) were added into the wells and incubated for 24 h. The peptide-conjugated plate were rinsed with Milli-Q™ water thoroughly and dried under argon flow.

### Antimicrobial Activity of BA-AMPs Coating Against Planktonic Bacteria

Bacterial strains used in this study included S. epidermidis (isolated from contaminated platelet units by Canadian Blood Services research laboratory in Ottawa), and S. saprophyticus (ATCC 15305). Bacteria were cultured in LB, with shaking at 250 rpm, at 37° C. for overnight. An overnight culture of the bacterial strain was diluted to a 500 CFU/mL in Mueller Hinton Broth (MHB). For antimicrobial activity experiments, 120 μL of the diluted overnight culture was added to each well in the bare PP plate, PP plate coated with BA and BA-AMPs. After 24 hrs growth under shaking condition at room temperature, bacteria culture (100 μL) was transferred to sterile clear polystyrene plate and the optical density was read using UV-vis spectrometer at wavelength 595 nm. Planktonic bacteria were then serially diluted and plated on LB agar for CFUs.

### Statistical Analysis

All the data values were expressed as mean±standard deviation (SD). Statistically significant value was set as p<0.05 based on Student's two-tailed unpaired t-test.

### EXAMPLES

### Example 1: Synthesis of Coatings with Different Structure and their Surface Characterization

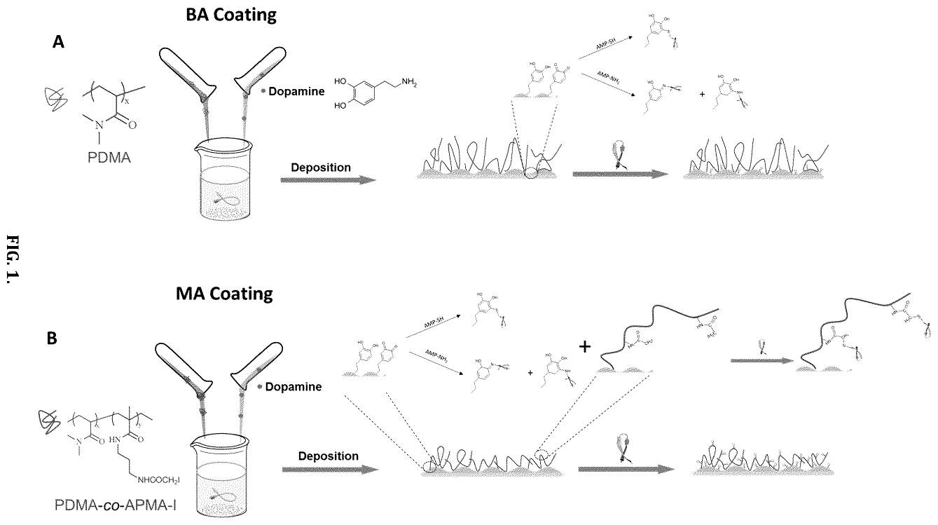

FIG. 1 shows the structure of the constructed two AMP conjugated coatings. Experiments were performed using a silicon wafer coated with Ti as a model surface for thorough characterization. AMPs with cysteine at the C-terminus was utilized for the conjugation into the coating. AMP E6 was used for initial investigation as it variant (without cysteine at the C-terminus) demonstrated strong broad-spectrum antimicrobial activity in soluble form^{[43-44]}. E6 in tethered form also showed strong anti-biofilm activity^{[13-14]}.

In the first approach, we initially constructed a non-fouling surface coating using a rapid assembly of polydopamine (PDA) and ultra-high molecular weight PDMA (uhPDMA) (800 KDa, PDI 1.3), which is highly stable and can be prepared on any substrate^{[39]}. The coating was highly hydrophilic and enriched with uhPDMA chains on the surface. The coating was further modified with AMP E6. The conjugation (FIG. 1A) utilized Michael-type addition reaction between the free —SH and —NH₂ groups on E6 and quinone functionality on PDA^{[45]}. The uhPDMA chains were not modified as it did not have any reactive functionalities. The anticipated structure of the coating is that peptide E6 conjugated to the bottom PDA layer protected by the non-fouling PDMA chains emanating from the PDA anchor. This approach is referred to as the BA-AMP coating throughout the manuscript since the AMP was attached to the bottom layer of the coating.

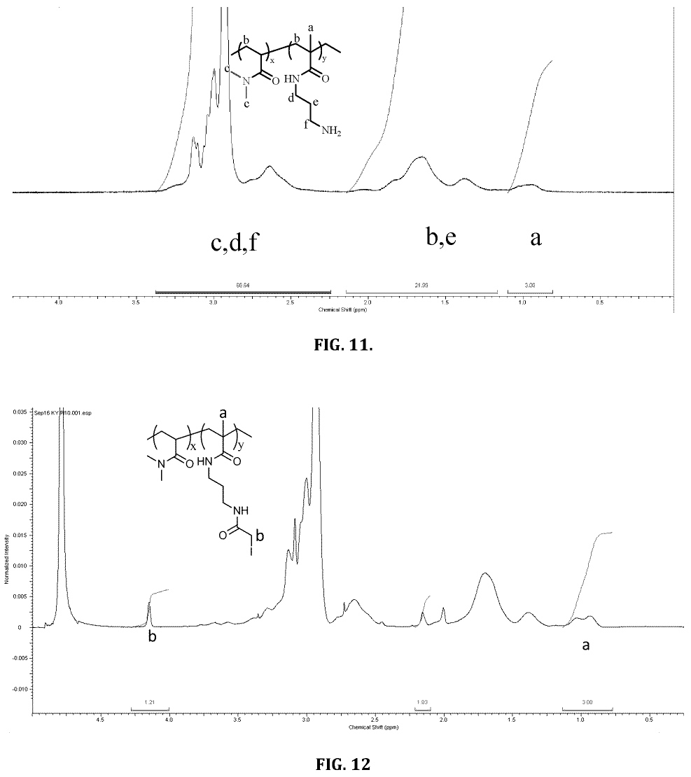

In the second approach, a copolymer PDMA-co-APMA (N-(3-aminopropyl) methacrylamide) (Mn 630 000, PDI L3) was synthesized with APMA molar content of 10% (FIG. 10). The copolymer was modified with iodoacetic acid (with a conversion of 60%, FIG. 11) to generate reactive groups for cysteine containing peptides on the polymer chains, and used for the generation of a non-fouling coating. PDMA-co-APMA-I was co-assembled with dopamine to generate a stable coating (FIG. 1B) which was then conjugated with AMP E6. The chemistry of the AMP conjugation is shown in FIG. 1B. Cysteine labeled E6 reacted with both iodoacetamide groups on the polymer chain and the quinone groups of PDA layer of the coating. The peptide was conjugated both to the hydrophilic polymer component as well as on the PDA layer. This resulted in the generation of a structure where the AMP was distributed throughout the coating. This is referred to as the MA-AMP coating throughout the manuscript since the AMPs are attached at multiple sites throughout the coating.

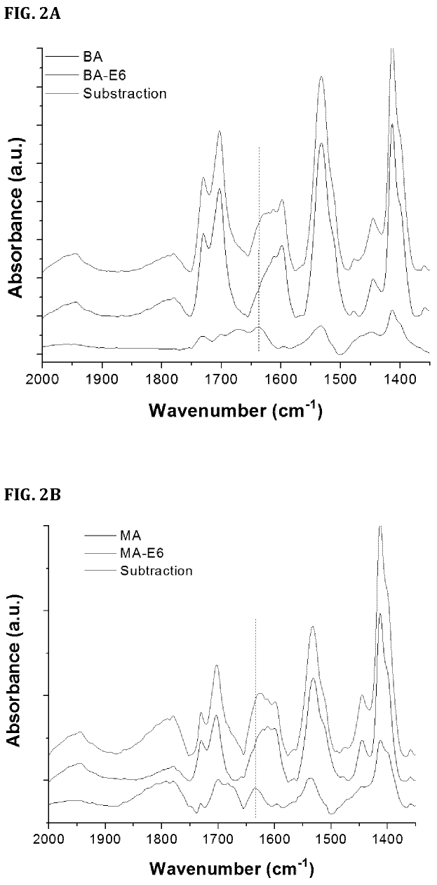

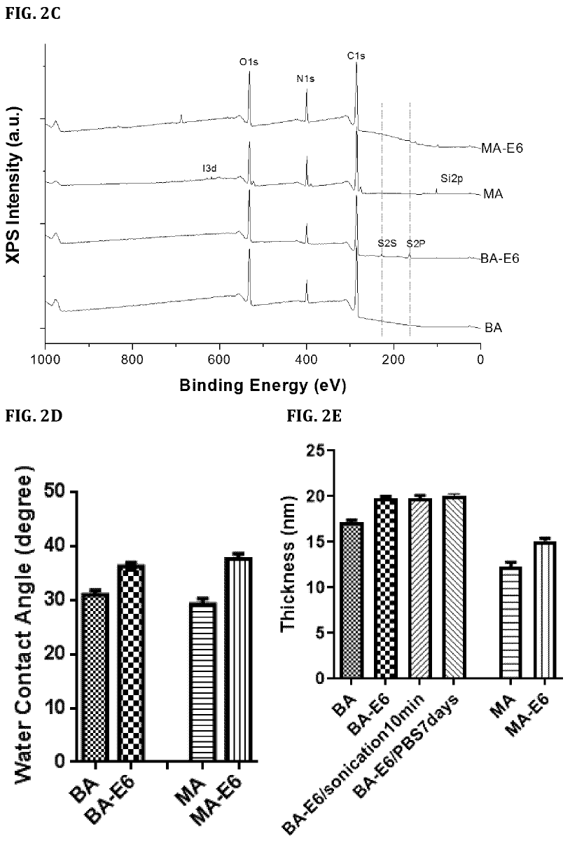

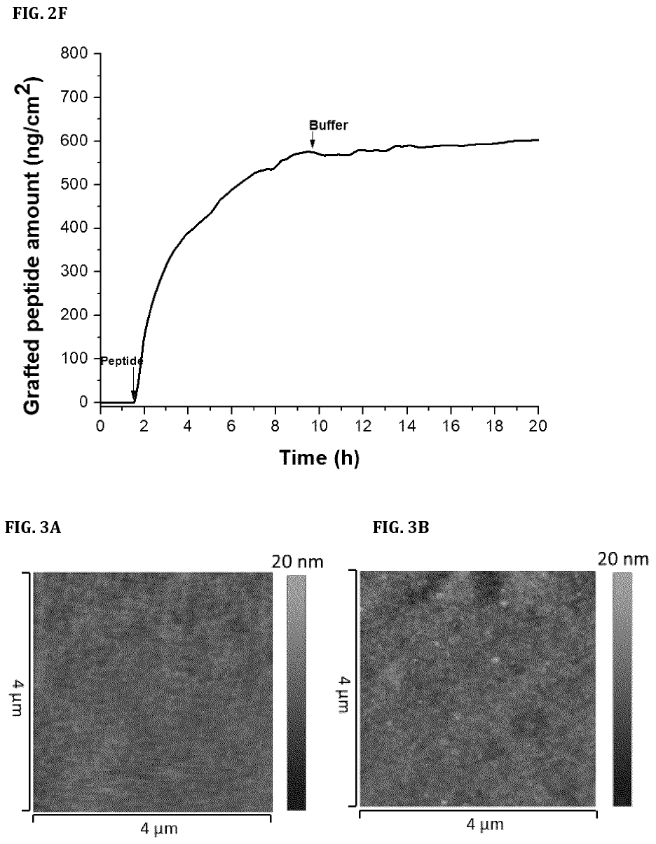

The coating formation and peptide conjugation were initially investigated using ATR-FTIR analysis (FIGS. 2A and 2B). The incorporation of uhPDMA and PDMA-co-APMA-I within the coating was evidenced by the shoulder peak at 1622 cm⁻¹ owing to the carbonyl group stretching (amide I band). The conjugation of AMP (E6) on the surface was demonstrated by the shoulder peak at 1634 cm⁻¹ on BA-AMP and MA-AMP coatings after AMPs conjugation. The subtraction spectra, before and after peptide conjugation, highlights the prominent peak at 1634 cm⁻¹ attributed to the C═O stretching for the amino acids in the AMPs, which confirmed the successful incorporation of AMPs in the coating. The incorporation of uhPDMA or APMA-co-APMA-I, and AMPs on the coating was further evidenced by the XPS analysis. The XPS survey scan (FIG. 2C) for the BA and BA-AMP coatings showed an increase in the nitrogen content in the coating compared to the PDA coating (TABLE 2). The presence of sulfur peak (S2p) (from cysteine residues) confirmed the presence of AMPs on the surface. In the case of the MA and MA-AMP coating, the presence of iodine(I3d) confirmed the successful incorporation of PDMA-co-APMA-I within the coating. The presence of S(2p) peak after AMPs (E6) conjugation, the disappearance of I(3d) peak in survey scan, and the change in surface elemental composition of the AMP modified surface (TABLE 2) confirmed the successful conjugation of E6 onto MA coating.

TABLE 2
Surface elemental composition of the coating
on silicon wafer substrate from XPS analysis

| | PDA | BA | BA-E6 | MA | MA-E6 |
|---|---|---|---|---|---|
| C % | 74.4 | 72 | 68.4 | 67.8 | 67.9 |
| N % | 6.9 | 9.7 | 9.9 | 10.8 | 13.7 |
| O % | 18.2 | 17.9 | 19.5 | 18.2 | 15.1 |
| Si % | 0.5 | 0.4 | 0 | 3.2 | 1.6 |
| S % | 0 | 0 | 2.2 | 0 | 1.8 |
| I % | 0 | 0 | 0 | 0.1 | 0 |

The conjugation of AMP to BA and MA coatings was further analyzed using water contact angle measurements. The water contact angle of BA-AMP coating was 36.2±0.4° in comparison to the BA coating 31±0.9°. In the case MA-AMP coating, the water contact angle was 37.9±0.9° compared to 29.2±1° for MA coating (FIG. 2D). Evidence for AMP conjugation was further obtained from the coating thickness measurements by ellipsometry. The dry thicknesses of BA and MA coatings were 17.2±0.2 and 12.3±0.5 nm, respectively. Upon conjugation of AMP E6 resulted in increases in thicknesses of 2.6±0.2 nm and 2.8±0.3 nm, respectively for the BA-AMP and MA-AMP coatings (FIG. 2E). The masses of grafted E6 on the BA and MA coating were 390±30 ng/cm² and 420±45 ng/cm² respectively. This calculation assumed that increase in thickness of the coating corresponded to the mass increase from the deposition of peptide E6^{[46]}. The grafting of E6 on to BA coating was further monitored in real-time using QCM-D to demonstrate the surface conjugation of the AMP (FIG. 2F). The amount of AMP E6 bound to the BA surface increased sharply in the first 2 h and reached a plateau after 8 h. The amount of AMP E6 remained on the surface was 568±72 ng/cm² after rinsing with buffer. The grafting density measured by QCM-D was higher than that measured by ellipsometry as the mass measured by QCM-D includes a substantial amount of bound water, whereas ellipsometry measured the conjugated AMP E6 mass in the dry state, and is consistent with previous work on mass quantification by QCM-D.⁴⁷ The coatings were further characterized for stability in buffer solutions; there was no noticeable change in thickness observed after immersing the BA-AMP coating in PBS buffer for 7 days at 37° C., or underwent ultrasonication for 10 min in water.

### Example 2: Evidences for Differences in Structure of Polymer Chains and Presentation of AMPs within the Coating by AFM Analysis

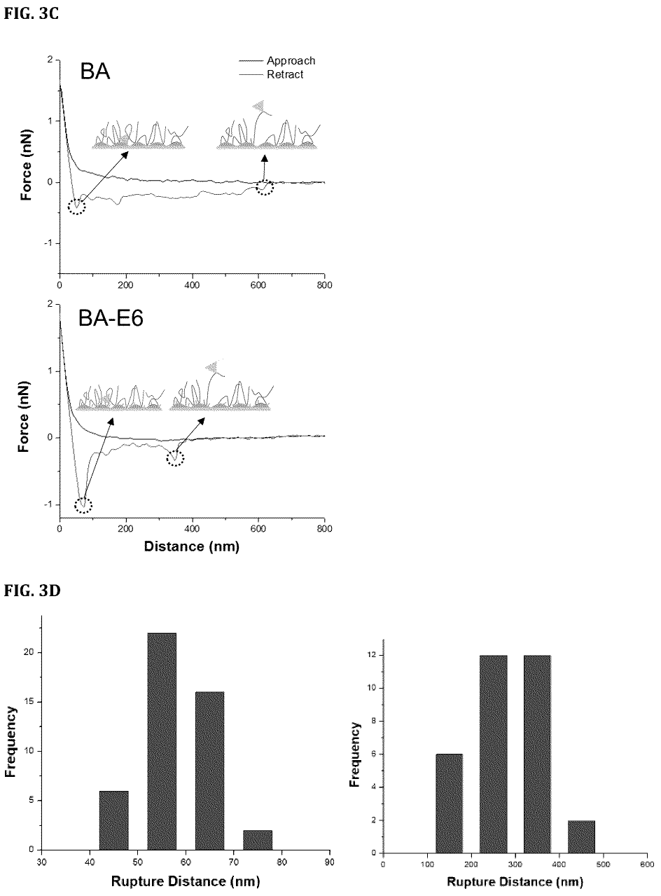

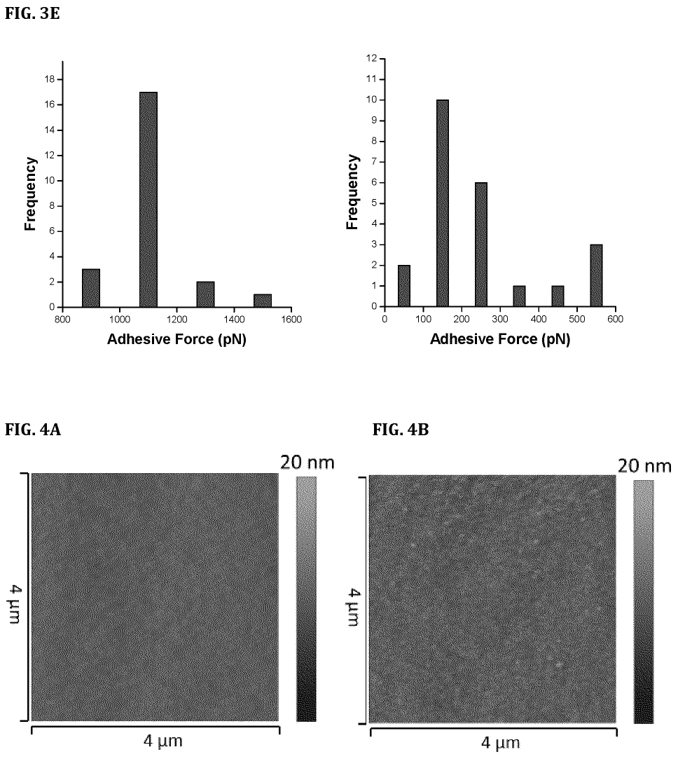

To gather the information on surface structure for two different coatings (BA-AMP and MA-AMP), we utilized AFM force measurements in wet conditions. The representative force curves for BA and BA-AMP coatings are shown in FIG. 3. AFM approach curves showed a typical force profile for steric repulsion exerted by polymer chains, reminiscent of swollen brush layer, grafted onto the surface for the BA-AMP (E6) coating (FIG. 3A). The AFM retraction curve for BA-AMP coating gave a characteristic profile of stronger adhesive force at shorter distances compared to the BA coating before AMP conjugation. FIGS. 3B and 3C show the probability distribution histograms of maximum adhesive force and rupture distance for BA-AMP (E6). The stronger force (1.1±0.1 nN) at shorter rupture distance (58.6±7.5 nm) could be due to the interaction between the peptide conjugated to the underlying PDA, and the hydrophobic AFM tip. This argument is supported by the fact that the retraction force curve for PDA-E6 coating (without the non-fouling uhPDMA chains) also showed similar characteristics in the probability distribution histograms with maximum adhesive force (2.2±0.2 nN) and rupture distance (47.4±5.3 nm) (FIG. 12). The smaller adhesive force (0.26±0.04 nN) at longer distance (306±100 nm) indicated the rupture of the adhered uhPDMA chains from the AFM tip. This data suggests that the PDMA chains on the surface might be adopting a looplike assembly^{[39]}. The data clearly showed that a swollen brush like structure due to the presence of uhPDMA chains was not completely lost and the chains offer steric protection even after AMP E6 conjugation. Together these data indicate that E6 was conjugated to the coating which was protected by the swollen uhPDMA layer.

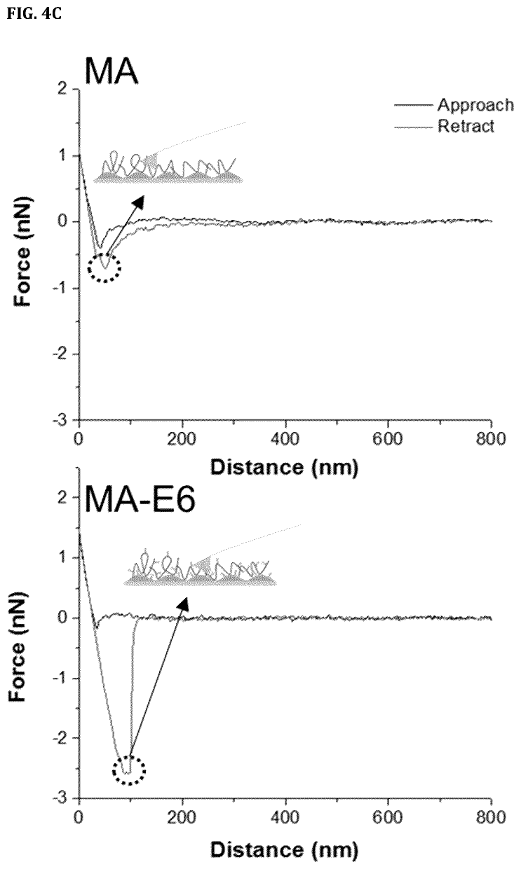

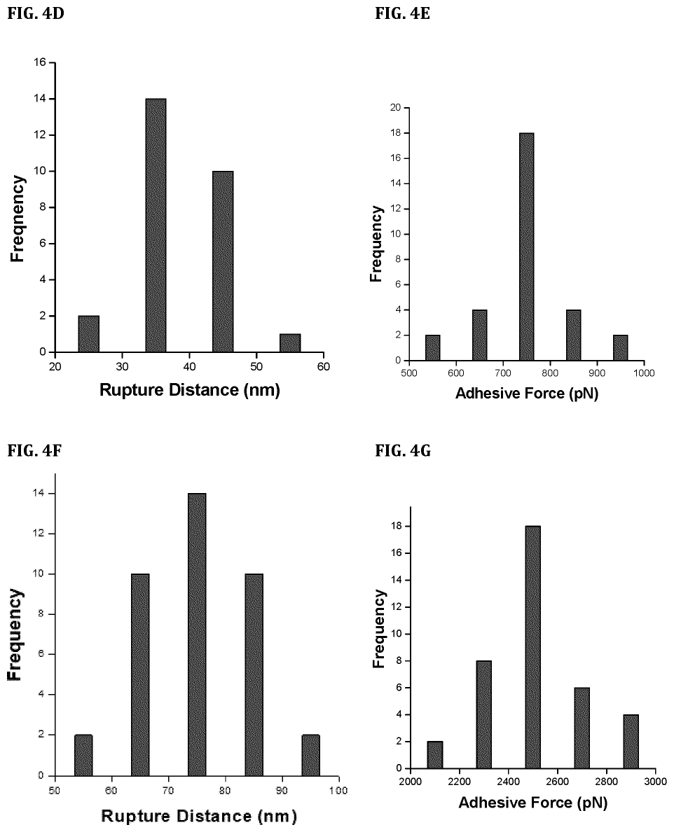

The representative AFM force curves for the MA and MA-AMP (E6) coatings are shown in FIG. 4. A weak jump-to-contact (JTC) force was observed when the AFM tip approached the surface for both MA and MA-AMP coatings. The data suggest that the copolymer modification resulted in a different surface assembly compared to BA and BA-AMP coatings^{[14, 46]}. During retraction, or pull-off, a stronger adhesive force 0.78±0.11 nN was observed in the case of the MA coating which increased to 2.6±0.2 nN when AMP E6 was conjugated. The strong adhesive force was due to the hydrophobic interaction between the AFM tip and the surface. The data indicated that in the case of the MA-AMP E6 coating, the more hydrophobic AMP E6 is presented on the outer layer without much steric protection from the copolymer resulting in strong hydrophobic interaction with the AFM tip. The probability distribution histograms of maximum adhesive force and rupture distance are shown in FIGS. 4B and 4C for MA and MA-AMP (E6), respectively. The weak jump-to-contact (JTC) force observed in the approach curve also indicated that the steric protection by the polymer chains in this structure might be low^{[14, 46]}. All these results pointed to a different structure of PDMA-co-APMA-I chains in the MA coating compared to PDMA chains in BA coating. There was no rupture observed at long distance, which suggests that the polymer chains were more confined onto the PDA layer and had less freedom to stretch out. Taken together, these data indicate that AMP E6 was present throughout the coating layer in the MA-AMP coating compared to the BA-AMP coating. The shielding ability of the non-fouling PDMA component was also relatively weak in the case of the MA-AMP coating.

The data clearly show that the structure of PDMA chains and presentation of AMPs (E6) were quite different for both BA-AMP and MA-AMP coatings. This allowed us to further investigate the role of presentation of AMP and coating structure to their anti-biofilm activity.

### Example 3: Anti-Biofilm Efficacy of AMP E6 Conjugated BA-AMP and MA-AMP Coatings

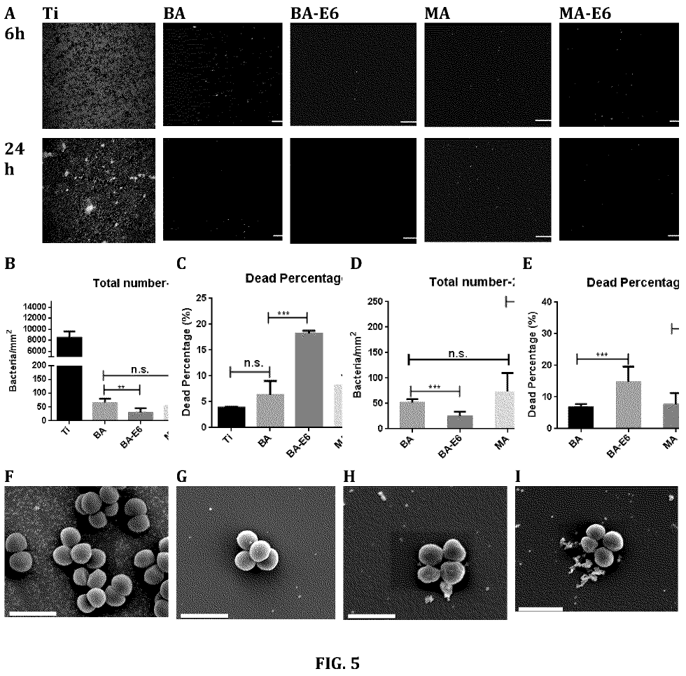

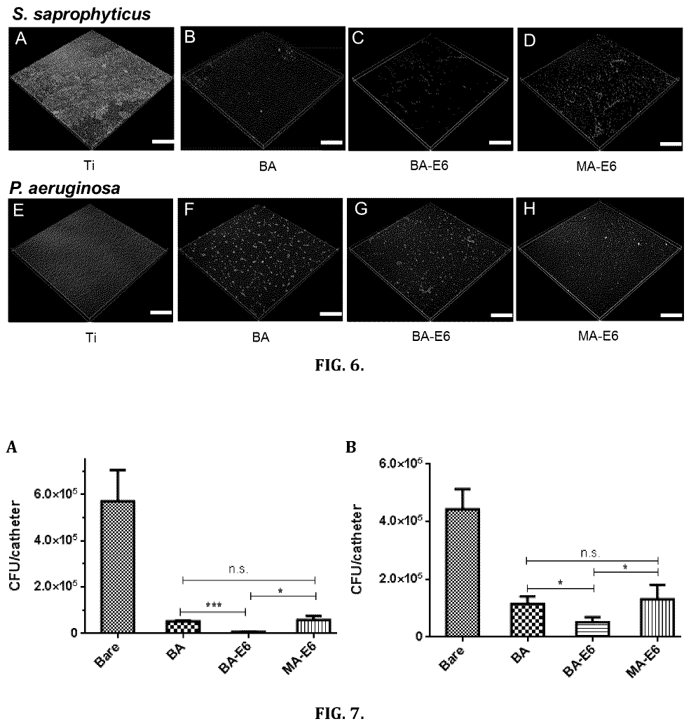

We further evaluated the influence of AMP presentation and coating structure on the anti-biofilm activity. A live/dead assay was used to examine the level of bacterial adhesion as well as the viability of adhered bacteria in early stage biofilm formation. FIG. 5 shows the number of adhered S. saprophyticus on different coatings after incubating with bacteria for 6 h and 24 h, as well as the proportion of dead bacteria. The BA-AMP (E6) was more resistant to bacterial adhesion when compared to the BA binary coating; the bacterial densities at 6 h were 57±12 and 29±15/mm² for the BA and BA-AMP coatings respectively. The MA coating showed similar activity in reducing bacterial adhesion compared to the BA coating; there were comparable numbers of adherent bacteria (66±15/mm²). In contrast, the MA-AMP surface had more bacteria adhered onto the surface (167±17/mm²). The percentages of dead bacteria were 6.3%, 18.2%, 8.2% and 18.3% for the BA, BA-AMP, MA, and MA-AMP coating respectively (FIG. 6C).

We further evaluated the early stage biofilm formation at 24 h. FIG. 5 and FIG. 6A show the fluorescence and confocal images of bacteria adhesion on bare and coated Ti substrates. FIGS. 5A and 6A showed that biofilm developed on the bare Ti substrate. There was a great reduction in biofilm formation on the BA and BA-AMP coated surfaces. Only a few bacterial colonies were observed on the BA-AMP coating (FIG. 6C) confirming the strong inhibition of biofilm formation. The BA and BA-AMP surfaces had similar numbers of adhered bacteria compared to 6 h (52±7/mm², 25±9/mm², respectively). In contrast, the MA and MA-AMP showed an increase (82±34/mm² and 191±51/mm²) in the number of adhered bacteria compared to 6 h. The AMP conjugated surfaces (BA-AMP and MA-AMP) sustained their antimicrobial activity as similar percentages of dead bacteria (14.8% and 19.7%) were recorded.

A similar observation was made for P. aeruginosa (FIG. 6). The BA-AMP (E6) coating showed greater efficiency in preventing bacterial colony formation on the surface. The activity was higher for the BA-AMP (E6) coating compared to MA-AMP (E6) suggesting that the presentation of AMP and coating structure influenced the anti-biofilm activity.

There are changes in membrane integrity and morphology upon interaction of the bacteria with the surface tethered AMPs on the coating. The bacteria on the control and BA coated substrates were observed to have a smooth surface (FIG. 5F, G), while those on BA-AMP and MA-AMP surfaces showed membrane collapse (FIG. 5H, I) and compromise of membrane integrity. This indicates that the surface-tethered AMPs generated using our coating interact with and kill bacteria via electrostatic disruption of the membrane.

### Example 4: Anti-Biofilm Efficacy of BA-AMP (E6) and MA-AMP (E6) Coating on Polyurethane Surface

We further analyzed the efficiency of the coating to prevent biofilm formation on a biomedical plastic surface relevant to catheter-associated urinary tract infections. We adapted the coating methodology on the surface of 14G PU catheters. The pristine catheter, BA, BA-AMP, MA and MA-AMP coating on PU catheters were tested by assessing the number of adhered bacteria on the surface by assessing CFUs after 24 h of incubation. FIG. 7 shows the adhesion of S. saprophyticus (A) and P. aeruginosa (B) on differently coated surfaces. The BA coating alone was able to reduce the adhesion of gram-positive S. saprophyticus by about 87.9% compared to the uncoated catheter which was consistent with previous reports^{[48]}. Upon conjugation of E6 (BA-AMP), the anti-biofilm activity of the coating increased to 98.4% (p<0.05). However, MA-AMP (E6) could not reduce the biofilm formation in comparison to the control coating without peptides or BA-AMP coatings. Similar results were observed for the gram-negative bacteria, P. aeruginosa.

From these results of bacterial adhesion and early stage biofilm formation, it was determined that presentation of AMPs on the coating and the coating structure had a significant influence on their anti-biofilm activity. The BA-AMP coating, where the AMP is conjugated on the PDA layer and protected by the non-fouling uhPMDA chains showed significantly higher activity than the coating where AMPs were presented throughout the coating (MA-AMP). Since the synthesis method for BA-AMP coating is very simple, we anticipate that this method could be easily and rapidly adapted to diverse surfaces and diverse peptides rapidly both as a screening method as well as an infection resistant coating.

### Example 5: Development of a Screening and Identification Method for Potent Surface Immobilized Anti-Biofilm Peptides in a Relevant Antifouling Background

To develop a robust screening protocol in a realistic environment which could be potentially used to test implant and device modifications, we initially tested a small library of AMPs peptides (TABLE 3) with broad-spectrum activity. Besides E6, Tet20C and its variant Tet20LC, IDR-1018, 3002, and D-enantiomeric peptide DJK5 and its variant, were also included in the study. Tet20C (KRWRIRVRVIRKC (SEQ ID NO:2)) and Tet20LC (KRWRIRVRVIRK-bA-bA-C—CONH2 (SEQ ID NO:3)) are variants of AMP Opt5 (KRWRIRVRVIRK-CONH2 (SEQ ID NO: 10)), which showed high antimicrobial activity in soluble and tethered form against various pathogens.

TABLE 3
Antimicrobial peptides, their sequences and minimal inhibition concentration (MIC)
used in this study. All of the tested peptides were amidated at the C-terminus
and lower case letters refer to D-amino acids.

| | | MIC (μg/mL) | | | | Grafting |
|---|---|---|---|---|---|---|
| | Sequence | P. | E. | S. | S. | density |
| AMP | (SEQ ID NO) | aeruginosa | coli | aureus | saprophyticus | (ng/cm²) |
| E6 | RRWRIVVIRVRRC | 32 | 8 | 64 | 4 | 312 ± 36 |
| | (SEQ ID NO: 1) | | | | | |
| Tet20C | KRWRIRVRVIRKC | 8 | 4 | 64 | 2 | 282 ± 42 |
| | (SEQ ID NO: 2) | | | | | |
| Tet20LC | KRWRIRVRVIRK- | 16 | 4 | 64 | 2 | 276 ± 36 |
| | bA-bA-C | | | | | |
| | (SEQ ID NO: 3) | | | | | |
| DJK5C | vqwrairvrvirc | 16 | 4 | 16 | 2 | 312 ± 12 |
| | (SEQ ID NO: 4) | | | | | |
| DJK5 | vqwrairvrvir | 16 | 1.6 | 16 | 2 | 222 ± 12 |
| | (SEQ ID NO: 5) | | | | | |
| IDR- | VRLIVAVRIWRR | 64 | 9.3 | 16 | 2 | 372 ± 12 |
| 1018 | (SEQ ID NO: 6) | | | | | |
| 3002C | ILVRWIRWRIQWC | 128 | 16 | 32 | 8 | 384 ± 24 |
| | (SEQ ID NO: 7) | | | | | |
| DJK6 | vqwrrirvwvir | | | | | |
| | (SEQ ID NO: 8) | | | | | |
| RI-DJK5 | rivrvrairwqv | | | | | |
| | (SEQ ID NO: 9) | | | | | |
| AMP | KRWRIRVRVIRK- | | | | | |
| Opt5 | CONH2 | | | | | |
| | (SEQ ID NO: 10) | | | | | |

Although the C-terminus of the tested peptides was amidated, modifications to the peptides are not a requirement for activity, but may have benefits to the activity of the AMP and assist in linking the AMP to the hydrophilic polymer or co-polymer. Adding a cysteine at C-terminus of Opt5 resulted in Tet20C. Tet20LC has a linker consisting of two beta-alanine added before the cysteine. Tet20C showed strong anti-biofilm activity while tethered in the brush coating on surface 1491. Peptide IDR-1018 was developed based on bactenecin and possessed both immunomodulatory activity and anti-biofilm activity^{[50]}. Peptide 3002 as a variant of IDR-1018, was discovered with computer aiding and exhibited stronger anti-biofilm activity than IDR-1018^{[51]}. DJK5 and its variants was recently developed D-enantiomeric protease-resistant peptide with a more potent activity in inhibiting biofilm formation relative to L-amino acid IDR-1018^{[52]}. This proven initial library of peptides (TABLE 3) was used to test our concept on the application of BA-AMP coating as a screening tool to identify optimal surface tethered peptide that prevents biofilm formation in an antifouling background.

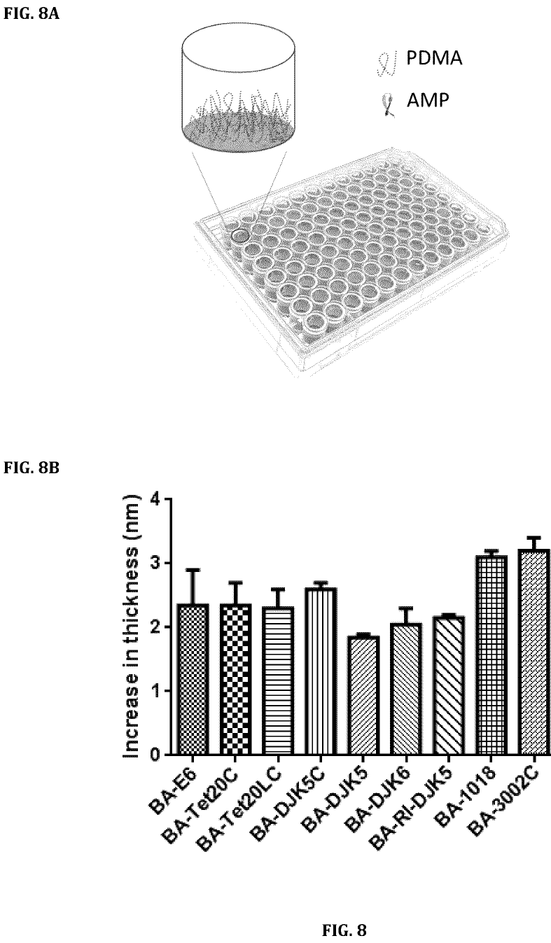

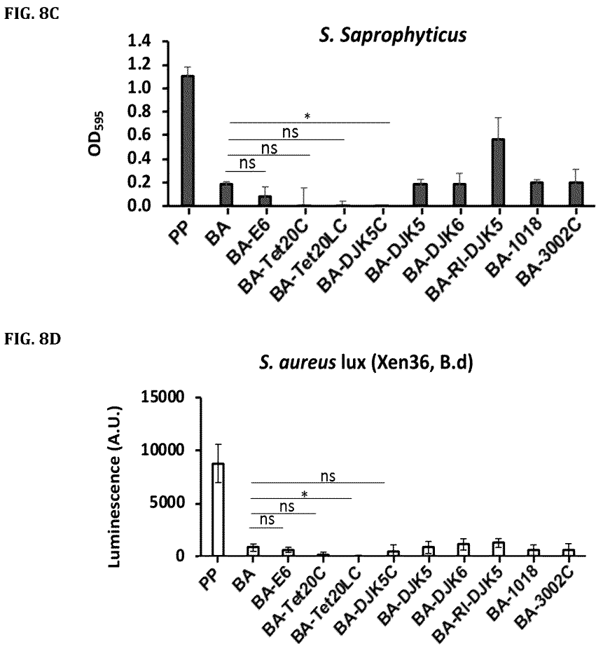

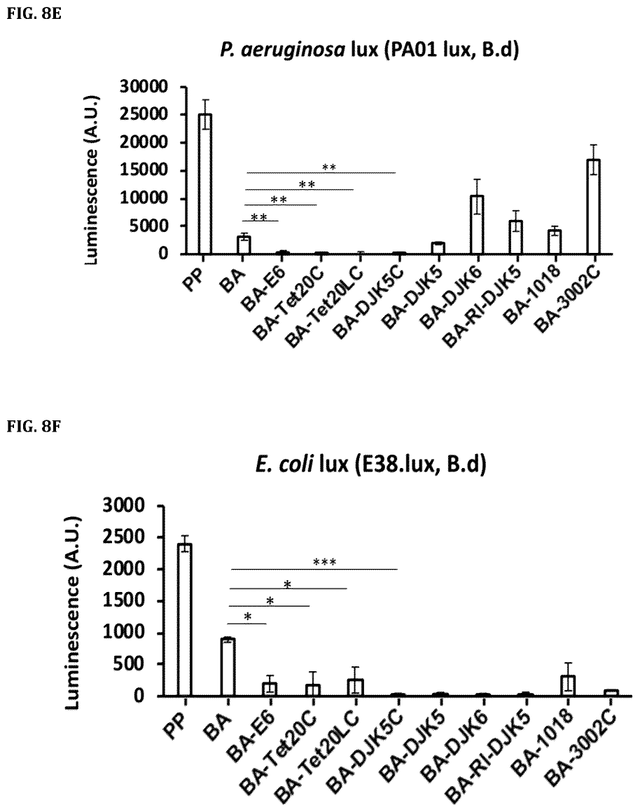

Initial studies were performed using a silicon wafer coated with titanium and the data showed that different AMPs could be successfully conjugated onto the BA coating (FIG. 8B). Based on this, we adapted the coating technique to a 96-well plate made of polypropylene (pp) for a medium-throughput screening of surface conjugated AMPs against diverse bacteria (FIG. 8A). We measured the bacterial adhesion on the BA coating and uncoated pp wells when compared to AMP conjugated wells. An anti-biofilm assay based on bioluminescence readout was developed to evaluate the efficiency of the coating and for identifying peptides with the best activity against biofilm formation. FIG. 8C shows the reduction of S. saprophyticus adhesion on different AMPs tethered on the BA coating upon incubation with bacteria over 24 h. The bioluminescence readout was confirmed with CFU counts in the case of the BA-AMP (E6) coating (FIG. 7). AMPs Tet20C, Tet20LC and DJK5C (containing as added cysteine at the C-terminus) showed almost complete reduction of biofilm formation (˜100%; p=0.002, p=0.01, p=0.003 respectively, pp vs BA-AMP). In contrast, AMPs DJK5, DJK6, IDR-1018 and 3002 showed no significant improvement over the BA coating (83.4%, p=0.98, 83.5%, p=0.97; 82.2%, p=0.71 and 81.7%, p=0.84 respectively). The peptide conjugated coating was also tested against clinically important S. aureus (FIG. 8D). Tethering E6 into the coating further reduced the biofilm formation (93%) compared to the BA coating (90.7%) (p=0.54, BA vs BA-E6). Near complete reduction was seen on the, Tet20C, Tet20LC conjugated surface (98.8%, p=0.003; and 100% p=0.002, pp vs BA-AMP). Surface conjugated with DJK5C, DJK5, IDR-1018, 3002C showed similar reduction levels compared to tethered E6 (95.1%, p=0.004; 90.5%, p=0.004; 93.5%, p=0.004; 93.6%, p=0.004; pp vs BA-AMP). We also investigated the activity against P. aeruginosa, which showed that tethering E6, Tet20C, Tet20LC and DJK5C resulted in the largest reduction of biofilm formation (98.2%, p=0.0002, 99.6%, p=0.0002, 100%, p=0.0002, and 99.1%, p=0.0002, respectively, pp vs BA-AMP) (FIG. 8E). DJK6, RI-DJK5, 1008 and 3002C have less biofilm reduction compared to BA coating (58.8%, 76.2%, 83.2% and 32.4%, respectively). Peptide conjugation also helped to provide additional reduction in E. coli biofilm formation (91.8% for tethered E6, p=0.008, pp vs BA-E6) (FIG. 8F). There was no significant difference in reduction efficiency observed for E. coli between the different peptides.

Overall, the 96-well-based anti-biofilm screening assay developed by conjugation of AMPs to a non-fouling background helped us to identify AMPs with better anti-biofilm activity. We determined that peptides, E6, Tet20C, Tet20LC and DJK5C showed better broad-spectrum activity than the other tested peptides from the library against the most common uro-pathogens.

### Example 6: Biocompatibility of BA-AMP Coating

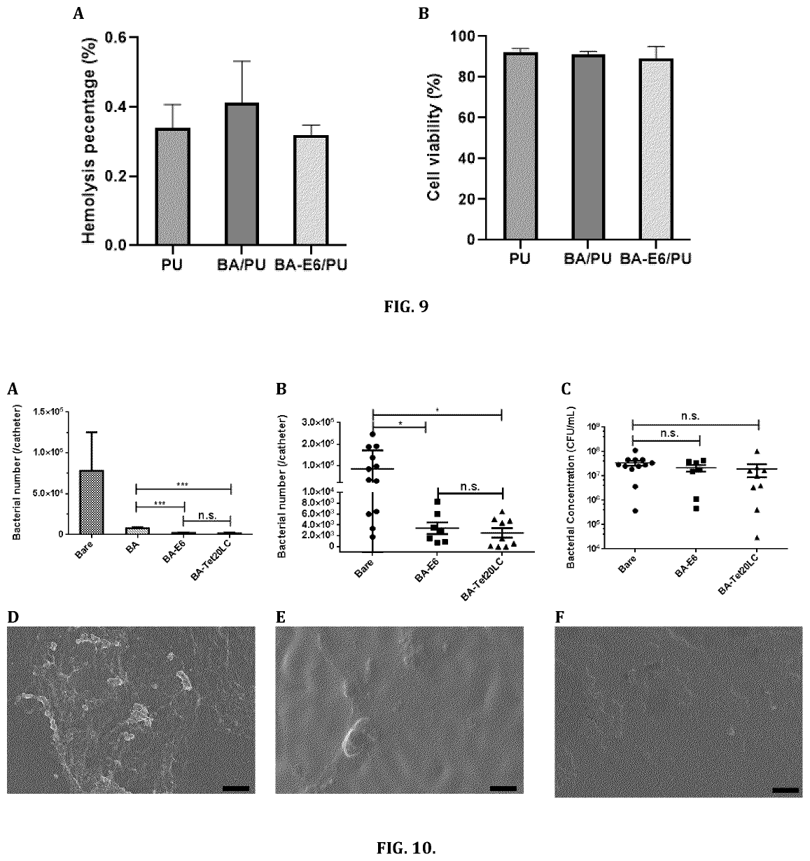

Hemolysis analysis was performed to determine the hemolytic properties of the AMP conjugated coating in direct contact with blood. The result of hemolysis analysis showed that the BA and BA-E6 coating has very low hemolysis, 0.4% and 0.3%, respectively (FIG. 9A). For the cyto-compatibility analysis, a cell viability assay utilizing using human bladder epithelial cells (T24) was performed. The viability of adhered cells on the PU substrate coated with BA and BA-AMP was very similar to those exposed to bare PU substrate (FIG. 9B). In both conditions, >90% cell viability was observed for both coated and uncoated substrates. Collectively these results suggest that BA-AMP coating has high biocompatibility.

### Example 7: Investigation of Anti-Biofilm Activity of Lead AMPs Tethered Coatings in a Mouse Urinary Infection Model

We next investigated whether our new coating chemistry and data from the screening studies could be utilized for the prevention of implant/device infection. We adapted the coating chemistry to 24G catheters using two different peptides that showed greater promise (BA-E6) and BA-Tet20LC). The generated coatings on catheters were studied for their in vitro activity first before testing in mouse infection models (FIG. 9A). The BA-E6 and BA-Tet20LC showed activity in preventing biofilm formation by 97.5% and 98.1%, respectively, compared to the 85% reduction seen on the control BA coating without peptides.

The efficiency in reducing biofilm formation was assessed using an ultrasound-guided percutaneous model of catheter-associated urinary tract infections^{[42]}. The reduction in bacterial numbers of S. saprophyticus both on the catheter sample as well as in the urine was accessed by CFU counts. The average CFU counts for mice bearing untreated control catheters was (3±0.7)×107 CFU/mL while those implanted with catheters bearing BA-AMP (E6) and BA-AMP (Tet20LC) coatings were (2.1±0.7)×107 and (1.8±0.9)×107 CFU/mL respectively, indicating that catheters from different groups were exposed to similarly infectious conditions (FIG. 9C). After 7 days post-instillation, the number of adhered bacteria on control catheters was (8.5±2.4)×104 CFU/catheter, whereas that for catheters coated with BA-E6 and BA-Tet20LC, was (3.4±1.1)×103 CFU/catheter, (2.5±0.8)×103 CFU/catheter, demonstrating a 96% and 97% reduction in bacterial adhesion respectively, when compared to the control (FIG. 9B).

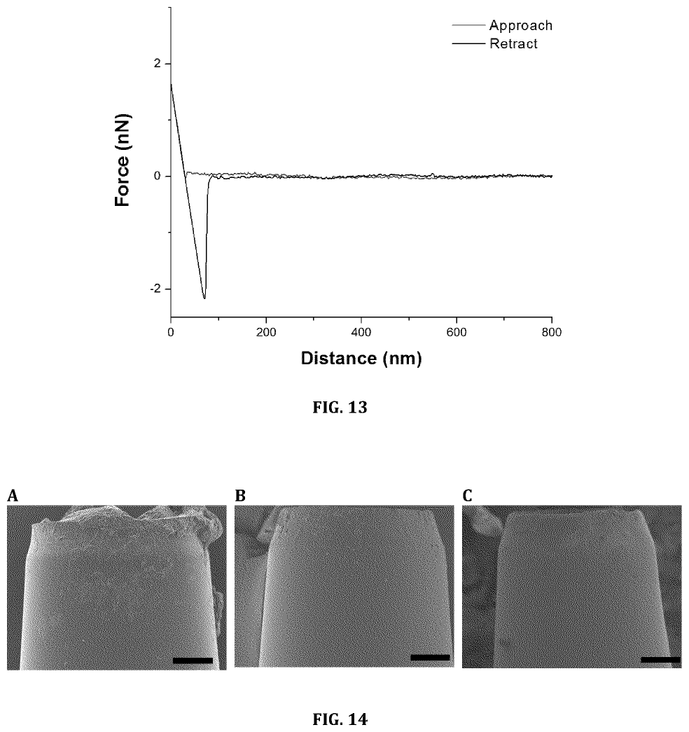

The biofilm formation on the catheter surface was also examined by scanning electron microscopy (SEM). FIGS. 9D, 9E, and 9F show the biofilm formation on the bare catheter and coated BA-AMP(E6) and BA-AMP (Tet20LC) catheters after 7 days post instillation. There was distinct biofilm formation on the bare catheter. The biofilm distributed along the catheter surface rather than covering the whole catheter piece (FIG. 9D, FIG. 13). We found a layer of extracellular matrix deposited on the bare catheter surface (FIG. 13). Biofilm was rarely seen on BA-E6 and BA-Tet20LC coated catheter (FIGS. 9E and 9F). A non-continuous layer of extracellular matrix was observed on coated catheter surfaces (FIG. 13).

### Example 8: Demonstration of Killing Activity of Surface-AMP Against Planktonic Bacteria

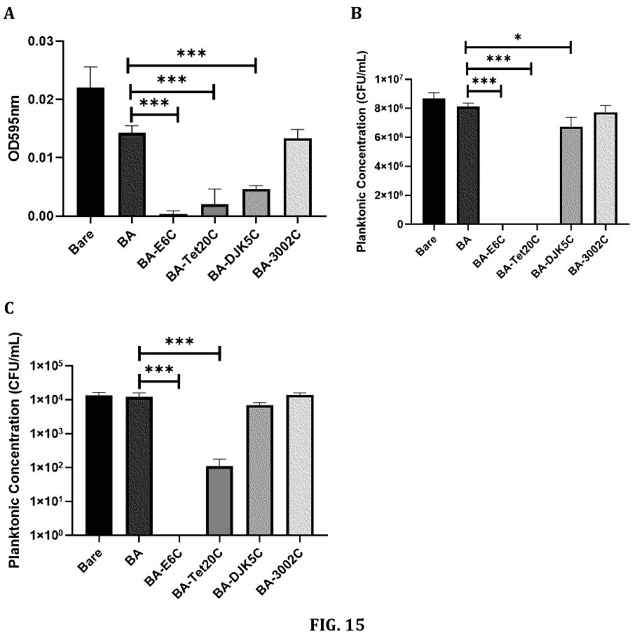

Conjugated peptides on the BA platform showed different inhibition capacities in association with planktonic bacteria growth and bacteria-killing activity (FIG. 15). Tethered E6 and Tet20 (with cysteine at the C-terminus—BA-E6C and BA-Tet20C) had the strongest antimicrobial activity among peptides with complete killing of S. saprophyticus in the MHB medium (FIGS. 15A and 14B). Tethered E6 also exhibited the strongest antimicrobial activity with complete killing of planktonic S. epidermidis (FIG. 15C). Compared to the bare (uncoated) wells, there was over 2 log reduction in the planktonic concentration of S. epidermidis cultured in the BA-Tet20C coated wells. The results demonstrate that BA-AMPs platform can be used as a high throughput platform for screening AMPs.

Although these results showed variable effects depending on the bacteria tested and the AMP used in the assay, effectiveness was shown for all AMPs tested for some bacteria. Accordingly, depending on the particular use (i.e. duration, location and microbiology of the individual, either the BA or MA polymer coating may be selected and the most suitable AMP or AMPs may be selected.

The disclosure may be further understood by the following non-limiting examples. Although the description herein contains many specific examples, these should not be construed as limiting the scope of the disclosure but as merely providing illustrations of some of the embodiments of the disclosure. For example, thus the scope of the disclosure should be determined by the appended aspects and their equivalents, rather than by the examples given.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this disclosure for a given application. In specific applications, the selection of a given anion or cation for preparation of a salt may result in increased or decreased solubility of that salt. Every formulation or combination of components described or exemplified herein may be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Unless otherwise explained, all technical and scientific terms used herein have the same meaning as commonly understood by one of ordinary skill in the art to which a disclosed disclosure belongs.

### REFERENCES

1. F. J. Xu, K. G. Neoh, E. T. Kang, Bioactive surfaces and biomaterials via atom transfer radical polymerization, Prog. Polym. Sci. 34 (2009) 719-761.
2. Q. A. Yu, Y. X. Zhang, H. W. Wang, J. Brash, H. Chen, Anti-fouling bioactive surfaces, Acta Biomater. 7 (2011) 1550-1557.
3. L Seon, P. Lavalle, P. Schaaf, F. Boulmedais, Polyelectrolyte Multi layers: A Versatile Tool for Preparing Antimicrobial Coatings, Langmuir 31(2015) 12856-12872.
4. X. Y. Zhu, X. J. Loh, Layer-by-layer assemblies for antibacterial applications, Biomater. Sci. 3 (2015) 1505-1518.
5. Y. Sui, X. L Gao, Z. N. Wang, C. J. Gao, Antifouling and antibacterial improvement of surface-functionalized poly(vinylidene fluoride) membrane prepared via dihydroxyphenylalanine-initiated atom transfer radical graft polymerizations, J. Membr.

### Sci. 394 (2012) 107-119.

6. C. H. Ho, J. Tobis, C. Sprich, R. Thomann, J. C. Tiller, Nanoseparated polymeric networks with multiple antimicrobial properties, Adv. Mater. 16 (2004) 957-961
7. W. He, Y. Zhang, J. H. Li, Y. L Gao, F. Luo, H. Tan, K. J. Wang, Q. Fu, A Novel Surface Structure Consisting of Contact-active Antibacterial Upper-layer and Antifouling Sub-layer Derived from Gemini Quaternary Ammonium Salt Polyurethanes, Sci Rep 6 (2016) 32140
8. X. Y. Zhang, Y. Q. Zhao, Y. D. Zhang, A. Z. Wang, X. K. Ding, Y. Li, S. Duan, X. J. Ding, F. J.

Xu, Antimicrobial Peptide-Conjugated Hierarchical Antifouling Polymer Brushes for Functionalized Catheter Surfaces, Biomacromolecules 20 (2019) 4171-4179.
9. Y. Y. He, X. Y. Wan, K. C. Xiao, W. W. Lin, J. H. Li, Z. Li, F. Luo, H. Tan, J. S. Li, Q. Fu, Anti-biofilm surfaces from mixed dopamine-modified polymer brushes: synergistic role of cationic and zwitterionic chains to resist Staphyloccocus aureus, Biomater. Sci. 7 (2019) 5369-5382.
10. B. Z. Wu, LX. Zhang, L Huang, S. W. Xiao, Y. Yang, M. Q. Zhong, J. Yang, Salt-Induced Regenerative Surface for Bacteria Killing and Release, Langmuir 33 (2017) 7160-7168.
11. T. Chen, H. Yang, X. Wu, D. F. Yu, A. Q. Ma, X. He, K. J. Sun, J. B. Wang, Ultrahighly Charged Amphiphilic Polymer Brushes with Super-Antibacterial and Self-Cleaning Capabilities, Langmuir 35 (2019) 3031-3037
12. T. N. Gevrek, K. Yu, J. N. Kizhakkedathu, A. Sanyal, Thiol-Reactive Polymers for Titanium Interfaces: Fabrication of Antimicrobial Coatings, ACS Appl. Polym. Mater. 1(2019) 1308-1316.
13. K. Yu, J. C. Y. Lo, Y. Mei, E. F. Haney, E. Siren, M. T. Kalathottukaren, R. E. W. Hancock, D. Lange, J. N. Kizhakkedathu, Toward Infection-Resistant Surfaces: Achieving High Antimicrobial Peptide Potency by Modulating the Functionality of Polymer Brush and Peptide, ACS Appl. Mater. Interfaces 7 (2015) 28591-28605.
14. K. Yu, J. C. Y. Lo, M. Yan, X. Q. Yang, D. E. Brooks, R. E. W. Hancock, D. Lange, J. N. Kizhakkedathu, Anti-adhesive antimicrobial peptide coating prevents catheter associated infection in a mouse urinary infection model, Biomaterials 116 (2017) 69-81.
15. Q. Xue, X. B. Liu, Y. H. Lao, L. P. Wu, D. Wang, Z. Q. Zuo, J. Y. Chen, J. Hou, Y. Y. Bei, X. F. Wu, K. W. Leong, H. Xiang, J. Han, Anti-infective biomaterials with surface-decorated tachyplesin I, Biomaterials 178 (2018) 351-362.
16. R. X. Chen, M. D. P. Willcox, K. K. K. Ho, D. Smyth, N. Kumar, Antimicrobial peptide melimine coating for titanium and its in vivo antibacterial activity in rodent subcutaneous infection models, Biomaterials 85 (2016) 142-151.
17. S. A. Onaizi, S. S. J. Leong, Tethering antimicrobial peptides: Current status and potential challenges, Biotechnol. Adv. 29 (2011) 67-74.
18. M. Salwiczek, Y. Qu, J. Gardiner, R. A. Strugnell, T. Lithgow, K. M. McLean, H. Thissen, Emerging rules for effective antimicrobial coatings, Trends Biotechnol. 32 (2014) 82-90.
19. H. Y. Ahmadabadi, K. Yu, J. N. Kizhakkedathu. Surface modification approaches for prevention of implant associated infections. Colloids and Surfaces B: Biointerfaces 193 (2020) 111116.
20. M. Godoy-Gallardo, C. Mas-Moruno, K. Yu, J. M. Manero, F. J. Gil, J. N. Kizhakkedathu, D.

Rodriguez, Antibacterial Properties of hLf1-11 Peptide onto Titanium Surfaces: A Comparison Study Between Silanization and Surface Initiated Polymerization, Biomacromolecules 16 (2015) 483-496.
21. H. Yazici, M. B. O'Neill, T. Kacar, B. R. Wilson, E. E. Oren, M. Sarikaya, C. Tamerler, Engineered Chimeric Peptides as Antimicrobial Surface Coating Agents toward Infection-Free Implants, ACS Appl. Mater. Interfaces 8 (2016) 5070-5081.
22. B. Mishra, A. Basu, R. R. Y. Chua, R. Saravanan, P. A. Tambyah, B. Ho, M. W. Chang, S. S. J. Leong, Site specific immobilization of a potent antimicrobial peptide onto silicone catheters: evaluation against urinary tract infection pathogens, J. Mat. Chem. B 2 (2014) 1706-1716.
23. G. P. Sakala, M. Reches, Peptide-Based Approaches to Fight Biofouling, Adv. Mater. Interfaces 5 (2018) 26.
24. T. Lou, X. Q. Bai, X. Y. He, C. Q. Yuan, Antifouling Performance Analysis of Peptide-Modified Glass Microstructural Surfaces. Appl. Surf. Sci. 541(2021) article No. 148384.
25. P. Cao, C. W. Du, X. Y. He, C. Zhang, C. Q. Yuan, Modification of a Derived Antimicrobial Peptide on Steel Surface for Marine Bacterial Resistance. Appl. Surf. Sci. 510 (2020) 145512.
26. P. Cao, K. W. Liu, X. D. Liu, W. Sun, D. L Wu, C. Q. Yuan, X. Q. Bai, C. Zhang, Antibacterial Properties of Magainin II Peptide onto 304 Stainless Steel Surfaces: A Comparison Study of Two Dopamine Modification Methods. Colloid Surf. B-Biointerfaces 194 (2020) article No. 111198.
27. K. Y. Lim, R. R. Y. Chua, H. Bow, P. A. Tambyah, K. Hadinoto, S. S. J. Leong, Development of a Catheter Functionalized by a Polydopamine Peptide Coating with Antimicrobial and Antibiofilm Properties. Acta Biomater. 15 (2015) 127-138.
28. D. W. Xu, W. H. Yang, Y. Hu, Z. Luo, J. H. Li, Y. H. Hou, Y. Liu, K. Y. Cai, Surface Functionalization of Titanium Substrates with Cecropin B to Improve Their Cytocompatibility and Reduce Inflammation Responses. Colloid Surf. B-Biointerfaces 110 (2013) 225-235.
29. X. W. Tan, T. W. Goh, P. Saraswathi, C. L Nyein, M. Setiawan, A. Riau, R. Lakshminarayanan, S. P. Liu, D. Tan, R. W. Beuerman, J. S. Mehta, Effectiveness of Antimicrobial Peptide Immobilization for Preventing Perioperative Cornea Implant-Associated Bacterial Infection. Antimicrob. Agents Chemother. 58 (2014) 5229-5238.
30. T. Shalev, A. Gopin, M. Bauer, R. W. Stark, S. Rahimipour, Non-leaching Antimicrobial Surfaces through Polydopamine Bio-inspired Coating of Quaternary Ammonium Salts or an Ultrashort Antimicrobial Lipopeptide. J. Mater. Chem. 22(2012) 2026-2032.
31. A. Saha, S. Nir, M. Reches, Amphiphilic Peptide with Dual Functionality Resists Biofouling, Langmuir 36 (2020) 4201-4206.
32. S. H. Mao, D. Zhang, X. M. He, Y. T. Yang, I. Protsak, Y. T. Li, J. W. Wang, C. X. Ma, J. Tan, J. T. Yang, Mussel-Inspired Polymeric Coatings to Realize Functions from Single and Dual to Multiple Antimicrobial Mechanisms. ACS Appl. Mater. Interfaces 13 (2021) 3089-3097.
33. K. Hilpert, M. Elliott, H. Jenssen, J. Kindrachuk, C. D. Fjell, J. Korner, D. F. H. Winkler, L. L Weaver, P. Henklein, A. S. Ulrich, S. H. Y. Chiang, S. W. Farmer, N. Pante, R. Volkmer, R. E. W. Hancock, Screening and Characterization of Surface-Tethered Cationic Peptides for Antimicrobial Activity. Chem. Biol. 16(2009) 58-69.
34. T. Wei, Z. C. Tang, Q. Yu, H. Chen, Smart Antibacterial Surfaces with Switchable Bacteria-Killing and Bacteria-Releasing Capabilities. ACS Appl. Mater. Interfaces 9 (2017) 37511-37523.
35. S. J. Yan, H. C. Shi, L. J. Song, X. H. Wang, L. Liu, S. F. Luan, Y. M. Yang, J. H. Yin, Nonleaching Bacteria-Responsive Antibacterial Surface Based on a Unique Hierarchical. ACS Appl. Mater. Interfaces 8(2016) 24471-24481.
36. H. Zhang, T. Zhao, B. Newland, W. Liu, W. Wang, W. Wang, Catechol functionalized hyperbranched polymers as biomedical materials, Prog. Polym. Sci. 78 (2018) 47-55.
37. D. R. Dreyer, D. J. Miller, B. D. Freeman, D. R. Paul, C. W. Bielawski, Perspectives on poly(dopamine), Chem. Sci. 4 (2013) 3796-3802.
38. M. E. Lynge, R. van der Westen, A. Postma, B. Stadler, Polydopamine-a nature-inspired polymer coating for biomedical science, Nanoscale 3 (2011) 4916-4928.
39. Y. Mei, K. Yu, J. C. Y. Lo, L. E. Takeuchi, N. Hadjesfandiari, H. Yazdani-Ahmadabadi, D. E. Brooks, D. Lange, J. N. Kizhakkedathu, Polymer-Nanoparticle Interaction as a Design Principle in the Development of a Durable Ultrathin Universal Binary Antibiofilm Coating with Long-Term Activity, Acs Nano 12 (2018) 11881-11891.
40. D. Pletzer, S. C. Mansour, R. E. W. Hancock, Synergy between conventional antibiotics and anti-biofilm peptides in a murine, sub-cutaneous abscess model caused by recalcitrant ESKAPE pathogens, Plos Pathogens 14 (2018) e10o7084.
41. E. F. Haney, M. J. Trimble, J. T. Cheng, Q. Valle, R. E. W. Hancock, Critical Assessment of Methods to Quantify Biofilm Growth and Evaluate Antibiofilm Activity of Host Defence Peptides, Biomolecules 8 (2018) 29.
42. C. Janssen, J. Lo, W. Jager, I. Moskalev, A. Law, B. H. Chew, D. Lange, A High Throughput, Minimally Invasive, Ultrasound Guided Model for the Study of Catheter Associated Urinary Tract Infections and Device Encrustation in Mice, J. Urol. 192(2014) 1856-1863.
43. K. Hilpert, R. Volkmer-Engert, T. Walter, R. E. W. Hancock, High-throughput generation of small antibacterial peptides with improved activity, Nat. Biotech. 23 (2005) 1008-1012.
44. K. Yu, B. F. L. Lai, J. Gani, R. Mikut, K. Hilpert, J. N. Kizhakkedathu, Interaction of blood components with cathelicidins and their modified versions, Biomaterials 69 (2015) 201-211
45. J. H. Ryu, P. B. Messersmith, H. Lee, Polydopamine Surface Chemistry: A Decade of Discovery, ACS Appl. Mater. Interfaces 10 (2018) 7523-7540.
46. G. Z. Gao, K. Yu, J. Kindrachuk, D. E. Brooks, R. E. W. Hancock, J. N. Kizhakkedathu, Antibacterial Surfaces Based on Polymer Brushes: Investigation on the Influence of Brush Properties on Antimicrobial Peptide Immobilization and Antimicrobial Activity, Biomacromolecules 12 (2011) 3715-3727.
47. P. Bingen, G. Wang, N. F. Steinmetz, M. Rodahl, R. P. Richter, Solvation Effects in the Quartz Crystal Microbalance with Dissipation Monitoring Response to Biomolecular Adsorption. A Phenomenological Approach, Anal. Chem. 80 (2008) 8880-8890.
48. K. Yu, A. Alzahrani, S. Khoddami, D. Ferreira, K. B. Scotland, J. T. J. Cheng, H. Yazdani-Ahmadabadi, Y. Mei, A. Gill, L. E. Takeuchi, E. Yeung, D. Grecov, R. E. W. Hancock, B. H. Chew, D. Lange, J. N. Kizhakkedathu, Self-Limiting Mussel Inspired Thin Antifouling Coating with Broad-Spectrum Resistance to Biofilm Formation to Prevent Catheter-Associated Infection in Mouse and Porcine Models, Advanced Healthcare Materials 10 (2021) 202001573.
49. G. Z. Gao, D. Lange, K. Hilpert, J. Kindrachuk, Y. Q. Zou, J. T. J. Cheng, M. Kazemzadeh-Narbat, K. Yu, R. Z. Wang, S. K. Straus, D. E. Brooks, B. H. Chew, R. E. W. Hancock, J. N. Kizhakkedathu, The biocompatibility and biofilm resistance of implant coatings based on hydrophilic polymer brushes conjugated with antimicrobial peptides, Biomaterials 32 (2011) 3899-3909.
50. S. C. Mansour, C. de la Fuente-Nunez, R. E. W. Hancock, Peptide IDR-1018: modulating the immune system and targeting bacterial biofilms to treat antibiotic-resistant bacterial infections, J. Pept. Sci. 21(2015) 323-329.
51. E. F. Haney, Y. Brito-Sanchez, M. J. Trimble, S. C. Mansour, A. Cherkasov, R. E. W. Hancock, Computer-aided Discovery of Peptides that Specifically Attack Bacterial Biofilms, Sci. Rep. 8 (2018) 1871.
52. C. de la Fuente-Nunez, F. Reffuveille, S. C. Mansour, S. L. Reckseidler-Zenteno, D. Hernandez, G. Brackman, T. Coenye, R. E. W. Hancock, D-Enantiomeric Peptides that Eradicate Wild-Type and Multidrug-Resistant Biofilms and Protect against Lethal Pseudomonas aeruginosa Infections, Chem. Biol. 22 (2015) 196-205.
53. Lei, J. et al. The antimicrobial peptides and their potential clinical applications, Am J Transl Res. (2019) 11(7): 3919-3931.
54. Moravej, H. et al. Antimicrobial Peptides: Features, Action, and Their Resistance Mechanisms in Bacteria, Microbial Drug Resistance (2018) 24(6):747-767.
55. Zhang, L-J. et al. Antimicrobial peptides, Current Biology (2016) 26(1):R14-R¹⁹.

What is claimed is:

1. A coating composition, the coating composition comprising:
(a) a polydopamine (PDA);
(b) a poly(N,N-dimethylacrylamide) (PDMA) polymer or a PDMA co-N-(3-Aminopropyl) Methacrylamide (APMA) polymer; and
(c) an antimicrobial peptide (AMP).

2. The coating composition of claim 1, wherein the PDMA polymer is either a high-molecular-weight (hPDMA) or an ultrahigh-molecular-weight (uhPDMA).

3. The coating composition of claim 1 or 2, wherein the PDMA co-APMA further comprises a linker.

4. The coating composition of claim 3, wherein the linker is selected from: an Iodoacetyl Linker (PDMA-co-APMA-I); a Bromoacetyl Linker (PDMA-co-APMA-Br); a Maleimide Linker (PDMA-co-APMA-M); and Pyridyl disulfide Linker (PDMA-co-APMA-Pd).

5. The coating composition of any one of claims 1-4, wherein the AMP is selected from one or more of: E6; Tet20C; Tet20LC; DJK5C; DJK5; DJK6; RI-DJK5; IDR-1018; and 3002C.

6. The coating composition of any one of claims 1, 2, 3 or 5, wherein the AMP is conjugated by an amine group or a thiol group to a quinone group on the PDA.

7. The coating composition of claim 4, wherein the thiol groups on the AMP are also conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer.

8. The coating composition of any one of claims 1-7, wherein the AMP is E6.

9. The coating composition of any one of claims 1-7, wherein the AMP is Tet20C.

10. The coating composition of any one of claims 1-7, wherein the AMP is Tet20LC.

11. The coating composition of any one of claims 1-7, wherein the AMP is DJK5C.

12. The coating composition of any one of claims 1-7, wherein the AMP is DJK5.

13. The coating composition of any one of claims 1-7, wherein the AMP is DJK6.

14. The coating composition of any one of claims 1-7, wherein the AMP is RI-DJK5.

15. The coating composition of any one of claims 1-7, wherein the AMP is IDR-1018.

16. The coating composition of any one of claims 1-7, wherein the AMP is 3002C.

17. The coating composition of any one of claims 1-16, wherein (b) is uhPDMA.

18. The coating composition of any one of claims 1-17, wherein the coating composition has an anti-fouling activity and an antimicrobial activity.

19. The coating composition of any one of claims 1-18, wherein the coating composition has anti-adhesion activity.

20. The coating composition of any one of claims 1-19, wherein the coating composition is for use in coating a medical device.

21. The coating composition of claim 20, wherein the medical device is for implantation in a subject.

22. A coated substrate, the coated substrate comprising:
(a) a substrate;
(b) a polydopamine;
(c) a PDMA polymer or a PDMA co-polymer; and
(d) an AMP.

23. The coated substrate of claim 22, wherein the substrate is selected from: a plastic; a metal; a ceramic; a carbon based material; a metal oxide; a hydrogels; a biological tissue; a wood; a cement; a rubber; a resin; and a composite.

24. The coated substrate of claim 22 or 23, wherein the substrate is selected from:
poly(propylene) (PP); poly(urethane) (PU); poly(ethylene) (PE); unplasticized polyvinyl chloride (uPVC); plasticized polyvinyl chloride (pPVC); poly(imide) (PI); ethylene vinyl acetate (EVA); poly(tetrafluoroethylene) (PTFE); polydimethylsiloxane (PDMS); polyisoprene(PIP); poly(N-hydroxymethyl acrylamide) (PHMA); poly(acrylamide) (PAM); poly(N-hydroxyethyl acrylamide) (PHEA); poly{N-[tris(hydroxymethyl) methyl]acrylamide} (PTHMAM); poly(methacrylamide) (PMA); poly(N-(2-hydroxypropyl)methacrylamide) (PHPMA); poly(vinyl pyrrolidone) (PVP); poly(ethylene oxide) (PEO); latex; titanium dioxide (TiO₂), titanium or silicon dioxide (SiO₂).

25. The coated substrate of claim 22, 23 or 24, wherein the substrate is PP, PU, PE, uPVC, pPVC, PI, EVA, or PTFE.

26. The coated substrate of any one of claims 22-25, wherein the substrate is TiO₂ or SiO₂.

27. The coated substrate of any one of claims 22-26, wherein the substrate forms part of an apparatus.

28. The coated substrate of any one of claims 22-27, wherein the apparatus is selected from:
a urinary device; a dental fixture; an artificial joint; a vascular device; a storage device;
blood storage device; a microfluidic device; a filtration membrane; a feed tube; or a diagnostic device.

29. The coated substrate of claim 28, wherein the vascular device is a catheter, a lead, or a stent.

30. The coated substrate of claim 28, wherein the urinary device is a urine storage device, blood storage device, catheter, or a stent.

31. The coated substrate of claim 28, wherein the filtration membrane is a blood filtration membrane, a water purification membrane, or an air purification membrane.

32. The coated substrate of claims 22-31, wherein the coated substrate reduces biofouling.

33. The coated substrate of claims 22-31, wherein the coated substrate reduces adhesion.

34. The coated substrate of claims 22-33, wherein the PDMA polymer is hPDMA or uhPDMA.

35. The coated substrate of claims 22-34, wherein the PDMA co-polymer is a copolymer of N,N-Dimethylacrylamide and N-(3-Aminopropyl) Methacrylamide with: an Iodoacetyl Linker (PDMA-co-APMA-I); a Bromoacetyl Linker (PDMA-co-APMA-Br); a Maleimide Linker (PDMA-co-APMA-M); or Pyridyl disulfide Linker (PDMA-co-APMA-Pd).

36. The coated substrate of claims 22-35, wherein the AMP is selected from one or more of: E6; Tet20C; Tet20LC; DJK5C; DJK5; DJK6; RI-DJK5; IDR-1018; and 3002C.

37. The coated substrate of claims 22-36, wherein the AMP is conjugated by an amine group or a thiol group to a quinone group on the PDA.

38. The coated substrate of claims 22-37, wherein the thiol groups on the AMP are also conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer.

39. A medical device including: a structure for implantation or disposition inside a subject, the structure including at least one surface for coating; wherein the at least one surface has a coating disposed directly on the at least one surface of the medical device, the coating comprising:
(a) a PDA;
(b) a PDMA polymer or a PDMA co-polymer; and
(c) an antimicrobial peptide (AMP).

40. A coating composition, the coating composition comprising:
(a) a PDA; and
(b) PDMA co-polymer.

41. The coating composition of claim 40, further comprising an antimicrobial peptide (AMP).

42. The coating composition of claim 40 or 41, wherein the PDMA polymer is hPDMA or uhPDMA.

43. The coating composition of claim 40, 41 or 42, wherein the PDMA co-polymer is a copolymer of N,N-Dimethylacrylamide and N-(3-Aminopropyl) Methacrylamide with: an Iodoacetyl Linker (PDMA-co-APMA-I); a Bromoacetyl Linker (PDMA-co-APMA-Br); a Maleimide Linker (PDMA-co-APMA-M); or Pyridyl disulfide Linker (PDMA-co-APMA-Pd).

44. The coating composition of any one of claims 41-44, wherein the AMP is selected from one or more of: E6; Tet20C; Tet20LC; DJK5C; DJK5; DJK6; RI-DJK5; IDR-1018; and 3002C.

45. The coating composition of any one of claims 41-44, wherein the AMP is conjugated by an amine group or a thiol group to a quinone group on the PDA.

46. The coating composition of any one of claims 41-44, wherein the thiol groups on the AMP are also conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer.

47. The coating composition of any one of claims 41-46, wherein the AMP is E6.

48. The coating composition of any one of claims 41-46, wherein the AMP is Tet20C.

49. The coating composition of any one of claims 41-46, wherein the AMP is Tet20LC.

50. The coating composition of any one of claims 41-46, wherein the AMP is DJK5C.

51. The coating composition of any one of claims 41-46, wherein the AMP is DJK5.

52. The coating composition of any one of claims 41-46, wherein the AMP is DJK6.

53. The coating composition of any one of claims 41-46, wherein the AMP is RI-DJK5.

54. The coating composition of any one of claims 41-46, wherein the AMP is IDR-1018.

55. The coating composition of any one of claims 41-46, wherein the AMP is 3002C.

56. The coating composition of any one of claims 40-55, wherein (b) is PDMA-co-APMA-I.

57. The coating composition of any one of claims 40-55, wherein (b) is hPDMA.

58. The coating composition of any one of claims 40-57, wherein the coating composition has an anti-fouling activity and an antimicrobial activity.

59. The coating composition of any one of claims 40-58, wherein the coating composition has anti-adhesion activity.

60. The coating composition of any one of claims 40-59, wherein the coating composition is for use in coating a medical device.

61. The coating composition of claim 60, wherein the medical device is for implantation in a subject.

62. A coating composition, the coating composition comprising:
(a) a PDA;
(b) a hydrophilic polymer co-APMA polymer; and
(c) an AMP;
wherein,
the hydrophilic polymer co-polymer has the structure of Formula I: 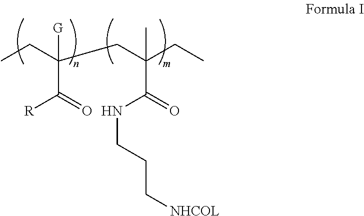
R is selected from 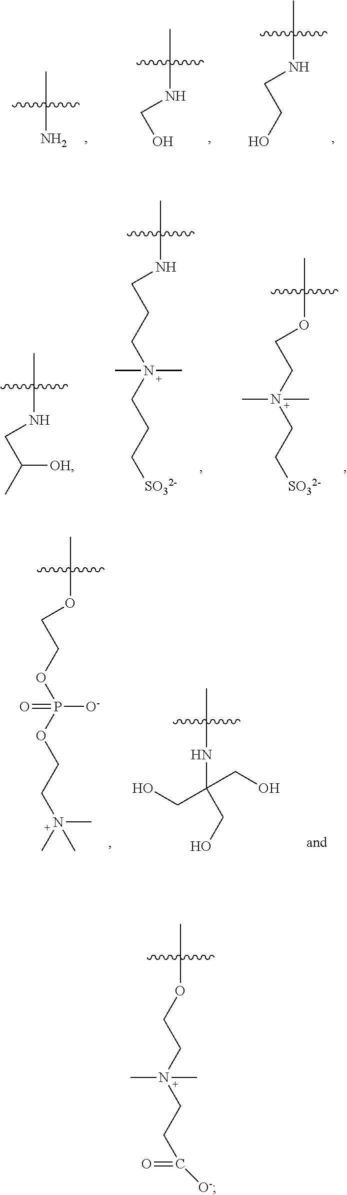
and
G is H or —CH₃;
L is a linking moiety;
n is an integer between woo and 20,000; and
m is an integer between 1 and 10,000.

63. The coating composition of claim 62, wherein the linker is selected from: CH₂I, CH₂Br,

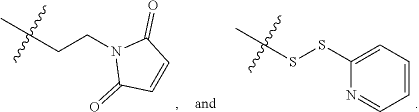 [SMILES: *CCN1C(=O)C=CC1=O ; *SSc1ccccn1]

64. The coating composition of claim 62 or 63, wherein hydrophilic polymer is copolymerized with: an Iodoacetyl Linker (co-APMA-I); a Bromoacetyl Linker (co-APMA-Br); a Maleimide Linker (co-APMA-M); or a Pyridyl disulfide Linker (co-APMA-Pd).

65. The coating composition of claim 62, 63 or 64, wherein hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

66. The coating composition of any one of claims 62-65, wherein the hydrophilic polymer is selected from PMPC and PMPDSAH.

67. The coating composition of claim 66, wherein the hydrophilic polymer co-APMA polymer is selected from PMPC-co-APMA-I and PMPDSAH-co-APMA-I.

68. The coating composition of any one of claims 62-67, wherein the AMP is E6.

69. The coating composition of any one of claims 62-67, wherein the AMP is Tet20C.

70. The coating composition of any one of claims 62-67, wherein the AMP is Tet20LC.

71. The coating composition of any one of claims 62-67, wherein the AMP is DJK5C.

72. The coating composition of any one of claims 62-67, wherein the AMP is DJK5.

73. The coating composition of any one of claims 62-67, wherein the AMP is DJK6.

74. The coating composition of any one of claims 62-67, wherein the AMP is RI-DJK5.

75. The coating composition of any one of claims 62-67, wherein the AMP is IDR-1018.

76. The coating composition of any one of claims 62-67, wherein the AMP is 3002C.

77. A substrate coating method, the method comprising:
(a) bringing the substrate into contact with a PDA and PDMA polymer or a PDMA co-APMA polymer solution;
(b) rinsing and drying;
(c) bringing the substrate into contact with an AMP solution;
(d) adding of a thiol containing hydrophilic compound; and
(e) rinsing and drying.

78. The substrate coating method of claim 77, further comprising a cleaning of the substrate prior to step (a).

79. The substrate coating method of claim 77 or 78, wherein the thiol containing hydrophilic molecule is selected from: 1-thioglycerol; thioethanol; 2-mercaptoethanol; 3-mercapto-1,2-propandiol; and dimercaptosuccinic acid.

80. The substrate coating method of claim 77, 78 or 79, wherein the bringing the substrate into contact with the PDA and PDMA polymer or a PDMA co-APMA polymer solution, is by immersing the substrate in the PDA and PDMA polymer or a PDMA co-APMA polymer solution.

81. The substrate coating method of any one of claims 77-80, wherein the bringing the substrate into contact with the AMP solution is by immersing the substrate in the AMP solution.

82. The substrate coating method of claim 81, wherein the immersing of the substrate in the AMP solution is for between 2-12 hours.

83. The substrate coating method of any one of claims 77-82, wherein following the addition of the thiol containing hydrophilic compound to the AMP solution, the substrate remains in the AMP solution with the thiol containing hydrophilic compound for between 12-24 hours.

84. The substrate coating method of any one of claims 77-83, wherein the rinsing in (b) and (e) is with water.

85. The substrate coating method of any one of claims 77-83, wherein the drying in (b) and (e) is under a stream of argon gas or a flow of nitrogen gas.

86. The substrate coating method of any one of claims 77-83, wherein the drying in (b) and (e) is under a stream of argon gas.

87. The substrate coating method of any one of claims 77-86, wherein the PDMA co-polymer is a copolymer of N,N-Dimethylacrylamide and N-(3-Aminopropyl) Methacrylamide with: an Iodoacetyl Linker (PDMA-co-APMA-I); a Bromoacetyl Linker (PDMA-co-APMA-Br); a Maleimide Linker (PDMA-co-APMA-M); or Pyridyl disulfide Linker (PDMA-co-APMA-Pd).

88. The substrate coating method of any one of claims 77-87, wherein the AMP is selected from one or more of: E6; Tet20C; Tet20LC; DJK5C; DJK5; DJK6; RI-DJK5; IDR-1018; and 3002C.

89. The substrate coating method of any one of claims 77-88, wherein the AMP is conjugated by an amine group or a thiol group to a quinone group on the PDA.

90. The substrate coating method of any one of claims 77-89, wherein the thiol groups on the AMP are also conjugated to one or more iodoacetamide groups on the PDMA-co-APMA-I polymer, one or more bromoacetamide groups on the PDMA-co-APMA-Br polymer, one or more maleimide groups on the PDMA-co-APMA-M polymer, or one or more 2-pyridyldithiol groups on the PDMA-co-APMA-Pd polymer.

91. A method of coating a substrate, wherein the substrate is immersed in a solution comprising the coating composition of any one of claims 1-21; 40-61; or 62-76.

92. A method of coating a substrate, wherein the substrate is sprayed with a solution or solutions comprising the composition of any one of claims 1-21; 40-61; or 62-76.

93. A coating composition, the coating composition comprising:
(a) a polymeric binder;
(b) a hydrophilic polymer, and
(c) an antimicrobial peptide (AMP);
wherein hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

94. The coating composition of claim 93, wherein the polymeric binder is selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin.

95. A coating composition, the coating composition comprising:
(a) a polymeric binder;
(b) a hydrophilic polymer co-APMA polymer; and
(c) an antimicrobial peptide (AMP);
wherein,
the hydrophilic polymer co-polymer has the structure of Formula I: 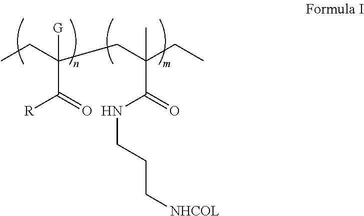
R is selected from 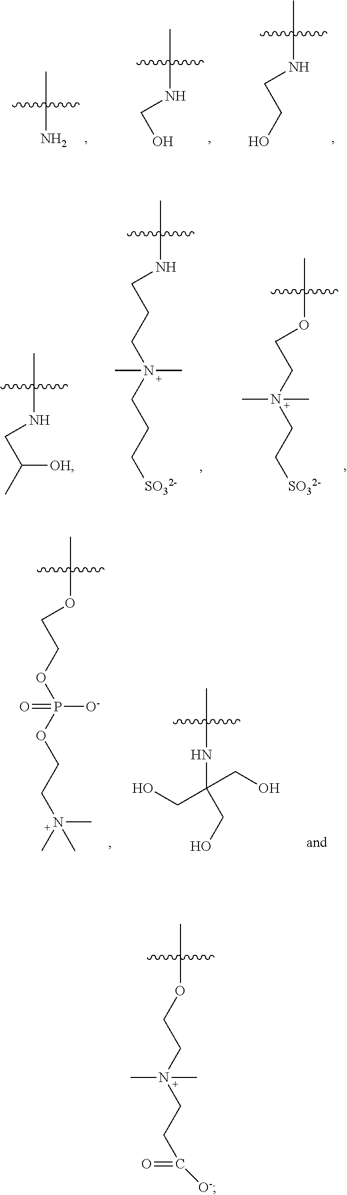
and
G is H or —CH₃;
L is CH₂I, CH₂Br, 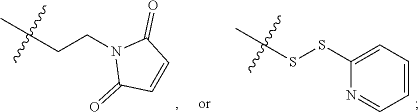 [SMILES: *CCN1C(=O)C=CC1=O ; *SSc1ccccn1]
n is an integer between 1000 and 20,000; and
m is an integer between 1 and 10,000.

96. The coating composition of claim 95, wherein the polymeric binder is selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin.

97. A substrate coating method, the method comprising:
(a) bringing the substrate into contact with a hydrophilic polymer and a polymeric binder solution;
(b) rinsing and drying;
(c) bringing the substrate into contact with an AMP solution;
(d) adding of a thiol containing hydrophilic compound; and
(e) rinsing and drying.

98. The substrate coating method of claim 97, wherein the polymeric binder is selected from one or more of: polymeric dopamine (PDA); polymeric norepinephrine (PNE); polymeric epinephrine (PEPI); polymeric pyrogallol (PPG); polymeric tannic acid (PTA); polymeric hydroxyhydroquinone (PHHQ); polymeric catechin; and polymeric epigallocatechin.

99. The substrate coating method of claim 97 or 98, wherein the hydrophilic polymer is selected from one or more of the following: polyacrylamide; poly(N-hydroxyethyl acrylamide); poly(N-(tris(hydroxymethyl)methyl) acrylamide); poly(N-(2-hydroxypropyl) methacrylamide); poly(N-hydroxymethyl acrylamide); poly((3-(methacryloylamino)propyl)dimethyl(3-sulfopropyl)ammonium hydroxide) (PMPDSAH); poly(2-methacryloyloxyethyl phosphorylcholine) (PMPC); poly(carboxybetaine methacrylate); and poly(sulfobetaine methacrylate).

100. The substrate coating method of claim 97, 98 or 99, further comprising a cleaning of the substrate prior to step (a).

101. The substrate coating method of any one of claims 95-100, wherein the thiol containing hydrophilic molecule is selected from: 1-thioglycerol; thioethanol; 2-mercaptoethanol; 3-mercapto-1,2-propandiol; and dimercaptosuccinic acid.


SEQUENCE LISTING (10 sequences)

SEQ ID NO: 1 - protein, 13 aa, Artificial Sequence
  E6 AMP
  MOD_RES (13)..(13): AMIDATION
RRWRIVVIRVRRC

SEQ ID NO: 2 - protein, 13 aa, Artificial Sequence
  Tet20C AMP
  MOD_RES (13)..(13): AMIDATION
KRWRIRVRVIRKC

SEQ ID NO: 3 - protein, 15 aa, Artificial Sequence
  Tet20LC AMP
  MOD_RES (13)..(13): bAla
  MOD_RES (14)..(14): bAla
  MOD_RES (15)..(15): AMIDATION
KRWRIRVRVIRKAAC

SEQ ID NO: 4 - protein, 13 aa, Artificial Sequence
  DJK5C d amino acids AMP
  MOD_RES (13)..(13): AMIDATION
VQWRAIRVRVIRC

SEQ ID NO: 5 - protein, 12 aa, Artificial Sequence
  DJK5 d amino acids AMP
  MOD_RES (12)..(12): AMIDATION
VQWRAIRVRVIR

SEQ ID NO: 6 - protein, 12 aa, Artificial Sequence
  IDR-1018 AMP
  MOD_RES (12)..(12): AMIDATION
VRLIVAVRIWRR

SEQ ID NO: 7 - protein, 13 aa, Artificial Sequence
  3002C AMP
  MOD_RES (13)..(13): AMIDATION
ILVRWIRWRIQWC

SEQ ID NO: 8 - protein, 12 aa, Artificial Sequence
  DJK6 d amino acids
  MOD_RES (12)..(12): AMIDATION
VQWRRIRVWVIR

SEQ ID NO: 9 - protein, 12 aa, Artificial Sequence
  AMP RI-DJK5 d amino acids AMP
  MOD_RES (12)..(12): AMIDATION
RIVRVRAIRWQV

SEQ ID NO: 10 - protein, 12 aa, Artificial Sequence
  Opt5 AMP
  MOD_RES (12)..(12): AMIDATION
KRWRIRVRVIRK